(19) United States
(12) Patent Application Publication
(10) Pub. No.: US 2015/0079078 A1
(43) Pub. Date: Mar. 19, 2015

(54) BIOMARKERS FOR TRIPLE NEGATIVE BREAST CANCER

(71) Applicant: Erasmus University Medical Center Rotterdam, Rotterdam (NL)

(72) Inventors: Azru Umar, Rotterdam (NL); Johannes Albert Foekens, Rotterdam (NL)

(21) Appl. No.: 14/391,495

(22) PCT Filed: Mar. 18, 2013
(86) PCT No.: PCT/NL2013/050197

§ 371 Date: Oct. 9, 2014

(30) Foreign Application Priority Data
Apr. 13, 2012 (NL) PCT/NL2012/050245

(51) Int. Cl. G01N 33/574; C12Q 1/68

(52) U.S. Cl. CPC G01N 33/57415; C12Q 1/6886; G01N 2800/52; G01N 2500/04
USPC 424/133.1; 435/6.12; 435/7.23; 506/7

(57) ABSTRACT

The present invention relates to biomarkers that are useful in the prognosis of triple negative breast cancer patients. The biomarkers may be used to select treatment and to determine whether a treatment is effective or not. The biomarkers may also be used to select novel treatments and to screen for new potential compounds that may treat the triple negative breast cancer.

The invention is directed to biomarkers for determining the prognosis of triple negative breast cancer. The invention is further related to determining the treatment and/or determining the effectiveness of a treatment in triple negative breast cancer as well as a screening method for compounds for triple negative breast cancer.

### BACKGROUND OF THE INVENTION

Breast cancer affects 1:8 women throughout their life and accounts for about 458,000 deaths worldwide annually. Tumour cells most commonly originate from epithelial cells lining the milk ducts or lobules. While histopathological parameters such as tumour grade, stage, and lymph node or distant metastasis have long been the golden standard to predict prognosis. Breast cancer is a very heterogeneous disease, consisting of different molecular subtypes. Molecular subtypes of breast cancer as defined by gene expression profiling were initially described a decade ago as biologically distinct disease entities with different clinical outcome.

The five main observed subtypes, luminal A, luminal B, HER2+, normal-like, and basal were named according to the expression of particular genes. The majority of breast tumors are of the luminal A subtype, which is characterized by, amongst others, high expression of estrogen receptor (ER) and progesterone receptor (PR), preferential metastasis to bone, and association with a relatively good prognosis. Luminal B type tumors have lower expression of ER and or PR, HER2+ tumors have an amplification of the human epidermal growth factor receptor 2 (HER2) gene, and normal-like and basal type tumors have high expression of basal epithelial cell type keratins, such as keratin 5 and 17, and are mostly characterized by the absence of ER, PR, and HER2. For that reason, the latter group is often referred to as ‘triple negative’.

A majority of breast tumors (˜80%) is ER, PR, or HER2+ positive and can be effectively treated with targeted therapies directed against these proteins, such as hormonal therapies blocking production or function of estrogens, and antibody or kinase inhibitor therapies blocking the HER2 pathway. A minority of the breast tumors, about 15%, are triple negative. Women with the triple negative subtype of breast cancer have poor prognosis and survival compared to other subtypes, due to the aggressive nature of these tumors and current absence of suitable targets for therapy. Triple negative tumors preferentially metastasize to lung and brain and have worst prognosis compared to other subtypes. An effective treatment for triple negative breast cancer is not readily available.

Despite a common triple negative phenotype, these tumours can clinically be defined as two separate groups based on disease prognosis. Within the triple negative subtype, 25% of the patients develop distant metastasis within 3 years, whereas 75% remains long-term metastasis-free.

Identification of biomarkers that can distinguish between these two classes of triple negative breast cancer may provide a fast and reliable prognosis and the basis for determination of an effective treatment. In addition, biomarkers that can distinguish between these two classes of triple negative breast cancer may provide development of new, targeted therapies against this aggressive type of breast cancer.

It is therefore an object of the present invention to provide biomarkers that are associated with triple negative breast cancer and preferably are able to determine the prognosis of triple negative breast cancer.

### SUMMARY OF THE INVENTION

In a first aspect the invention relates to a method for determining a prognosis for a patient with triple negative breast cancer comprising determining a level of expression of biomarker AP1G1 and/or CAPZB in a biological sample from said patient.

In another aspect and/or preferred embodiment of the present invention the method further comprises determining the expression level of at least one biomarker selected from the group comprising CMPK1, PRKACA, EML4, GANAB, PSME2, PRKAR1A, FTH1, MDH1, OTUB1, TF, DPYSL2, MGP, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1.

In another aspect and/or preferred embodiment of the present invention the method further comprises determining the expression level of at least one biomarker selected from the group comprising MTHFD1, CTNNA1, STX12, AP1M1,

The expression of said biomarker may be up-regulated or down regulated.

The expression of AP1G1 and/or CAPZB is downregulated in said sample correlates with poor prognosis of said patient.

The expression of CTNNA1, STX12, and/or AP1M1 is down-regulated in said sample correlates with poor prognosis of said patient.

The expression of MTHFD1 is upregulated in said sample correlates with poor prognosis of said patient.

The level of expression of at least one biomarker selected from the group consisting of PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, UBE2Q1, SMC4, and/or HAPLN1, is up-regulated and/or the level expression of at least one biomarker selected from the group consisting of CMPK1, PRKACA; PRKACB, EML4, GANAB, PSME2, PRKAR1A, FTH1, MDH1, OTUB1, TF, DPYSL2, MGP, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM is down-regulated in said sample correlates with poor prognosis of said patient.

The expression of AP1G1 and/or CAPZB is upregulated in said sample correlates with increased survival of said patient.

The expression of CTNNA1, STX12, and/or AP1M1 is upregulated in said sample correlates with increased survival of said patient.

The expression of MTHFD1 is downregulated in said sample correlates with increased survival of said patient.

The level expression of at least one biomarker selected from the group consisting of PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, UBE2Q1, SMC4, and/or HAPLN1 is down-regulated and/or the level expression of at least one biomarker selected from the group consisting of CMPK1, PRKACA; PRKACB, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM is up-regulated in said sample correlates with increased survival of said patient.

Another aspect of the invention relates to the use of protein or nucleic acid coding for protein selected from group consisting of AP1G1 and/or CAPZB as biomarker to determine prognosis in triple negative breast cancer.

In a preferred embodiment of the invention relates to the use of protein or nucleic acid coding for protein selected from group consisting of CTNNA1, STX12, MTHFD1, and/or AP1M1 as biomarker to determine prognosis in triple negative breast cancer.

Another aspect and/or embodiment of the invention relates to the use of protein or nucleic acid coding for protein selected from group consisting of CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, K1AA0174, HLA-C, UBE2Q1, PSMB9, AGL, GOSR1, PTK2, SMC4, HAPLN1, SKIV2L, and/or GSTM1 as biomarker to determine prognosis in triple negative breast cancer. The prognosis may be poor or increased survival.

Yet another aspect of the invention relates to a method of determining effectiveness of treatment for a patient with triple negative breast cancer comprising determining at a first time point the level of expression at least one biomarker selected from the group comprising AP1G1 and/or CAPZB in a biological sample from said patient and determining at a second time point the level of expression at least one biomarker selected from the group comprising AP1G1 and/or CAPZB in a biological sample from said patient.

Yet another aspect and/or embodiment of the invention relates to a method of determining effectiveness of treatment for a patient with triple negative breast cancer comprising determining at a first time point the level of expression at least one biomarker selected from the group comprising CTNNA1, STX12, MTHFD1, and/or AP1M1 in a biological sample from said patient and determining at a second time point the level of expression at least one biomarker selected from the group comprising CTNNA1, STX12, MTHFD1, and/or AP in a biological sample from said patient.

Yet another aspect and/or embodiment of the invention relates to a method of determining effectiveness of treatment for a patient with triple negative breast cancer comprising determining at a first time point the level of expression at least one biomarker selected from the group comprising CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDCl₂₃, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1 in a biological sample from said patient and determining at a second time point the level of expression at least one biomarker selected from the group comprising CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, K1AA0174, HLA-C, UBE2Q1, PSMB9, AGL, GOSR1, PTK2, SMC4, HAPLN1, SKIV2L, and/or GSTM1 in a biological sample from said patient.

Yet the invention relates in another aspect of the invention to a method of determining treatment for a patient with triple negative breast cancer comprising determining a level of expression of at least one biomarker selected from the group comprising AP1G1 and/or CAPZB in a biological sample from said patient.

Yet the invention relates in another aspect and/or embodiment of the invention to a method of determining treatment for a patient with triple negative breast cancer comprising determining a level of expression of at least one biomarker selected from the group comprising MTHFD1, CTNNA1, STX12, and/or AP1M1 in a biological sample from said patient.

Yet the invention relates in another aspect and/or embodiment of the invention to a method of determining treatment for a patient with triple negative breast cancer comprising determining a level of expression of at least one biomarker selected from the group comprising CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1 in a biological sample from said patient.

A further aspect of the invention relates to a method to screen for compounds for treatment of triple negative breast cancer using at least one biomarker selected from the group consisting of AP1G1 and/or CAPZB.

A further aspect and/or embodiment of the invention relates to a method to screen for compounds for treatment of triple negative breast cancer using at least one biomarker selected from the group consisting of MTHFD1, CTNNA1, STX12, and/or AP1M1

A further aspect and/or embodiment of the invention relates to a method to screen for compounds for treatment of triple negative breast cancer using at least one biomarker selected from the group consisting of CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1.

Furthermore another aspect of the invention relates to a kit for determining a prognosis, a treatment, and/or the effectiveness of a treatment for a patient with triple negative breast cancer, wherein said kit comprises a compound capable of detecting the level of expression of at least one biomarker selected from the group of AP1G1 and/or CAPZB in a biological sample.

Furthermore another aspect and/or embodiment of the invention relates to a kit for determining a prognosis, a treatment, and/or the effectiveness of a treatment for a patient with triple negative breast cancer, wherein said kit comprises a compound capable of detecting the level of expression of at least one biomarker selected from the group of MTHFD1, CTNNA1, STX12, and/or AP1M1 in a biological sample.

Furthermore another aspect and/or embodiment of the invention relates to a kit for determining a prognosis, a treatment, and/or the effectiveness of a treatment for a patient with triple negative breast cancer, wherein said kit comprises a compound capable of detecting the level of expression of at least one biomarker selected from the group of CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1 in a biological sample.

### DESCRIPTION OF THE FIGURES

FIG. 1: Kaplan Meier curves biomarker set CMPK1, AIFM1, FTH1, EML4, GANAG, AP1G1, and CAPZB.

FIG. 2: Kaplan Meier curves biomarker set EML4, AP1G1, STX12, and CAPZB.

FIG. 3: Kaplan Meier curves biomarker set with EML4, AP1G1, and CAPZB.

FIG. 4: Kaplan Meier curves biomarker set with CMPK1, AIFM1, FTH1, AP1G1, AP1M1, CAPZB.

FIG. 5: Kaplan Meier curves biomarker set with CMPK1, AIFM1, FTH1, AP1G1, CAPZB.

FIG. 6: Kaplan Meier curves biomarker set with markers AP1G1 and CAPZB.

FIG. 7: Kaplan Meier curves biomarker set CMPK1, AIFM1, FTH1, EML4, and GANAG.

FIG. 8: Kaplan Meier curves biomarker set EML4 and STX12

### DEFINITIONS

For the purpose of the present invention, a biomarker may be a protein or nucleic acid coding for protein, a peptides or a metabolite. Preferred biomarkers according to the invention and/or embodiments thereof are proteins, peptides, or nucleic acids coding for a protein. Most preferred biomarkers according to the invention and/or embodiments thereof are proteins or peptides, and/or fragments of the protein and/or peptides.

The present invention and embodiments thereof is directed to biomarkers that may be detected in a biological sample. Biological sample may be selected for the group consisting of breast tissue, blood, lymph fluid, serum, urine, circulating cancer cells, and/or nipple aspirate.

For the present invention, poor prognosis is defined as developing distant metastasis within 5 year after diagnosis.

Good prognosis is defined as being metastasis free after 5 years after diagnosis.

Increased survival rate is based on Kaplan Meier survival curve for progression and/or metastasis free survival. The Kaplan-Meier estimator also known as the product limit estimator is an estimator for estimating the survival function from life-time data. In medical research, it is often used to measure the fraction of patients living for a certain amount of time after treatment. A plot of the Kaplan-Meier estimate of the survival function is a series of horizontal steps of declining magnitude which, when a large enough sample is taken, approaches the true survival function for that population. The value of the survival function between successive distinct sampled observations (“clicks”) is assumed to be constant. 95% of patients with ‘good’ profile stay metastasis free for more than 10 years, whereas about 70% of patients with ‘poor’ profile have metastasis within 2 years.

Patient in the present invention is a patient diagnosed with triple negative breast cancer. Triple negative breast cancers are cancers that don't have receptors for estrogen, progesterone or human epidermal growth factor (Her2). Triple negative breast cancer is denoted (ER-), (PR-), (HER2-). Often a biopsy is taken to test for these receptors. Several assays are known that can determine the presence or absence of ER, PR and HER2, such as e.g. fluorescent assay, and/or immunohistochemical assay. Preferably the method and markers of the present invention and/or embodiments thereof are used after diagnosis of triple negative breast cancer is made.

Triple-negative breast cancer is typically treated with a combination of therapies such as surgery, radiation therapy, and chemotherapy. Some research has shown that hormone-receptor-negative breast cancers—which triple-negative breast cancers are—actually respond better to a combination of chemotherapy than breast cancers that are hormone-receptor-positive. At this time, however, there is no standard recommendation for people with triple-negative breast cancer. Researchers are currently studying various types of biological therapy including olaparib, a PARP-1 inhibitor.

Surgery for Triple Negative Breast Cancer Treatment. Depending on where the cancer is located in the breast and how large in size it is doctors may decide to perform one of two types of surgeries. The first, referred to as breast-conserving surgery (or a lumpectomy or partial mastectomy), occurs when a surgeon only removes the area of the breast that is affected by the cancer. The second, known as a mastectomy, is where the surgeon removes the entire breast. During each of these two types of surgeries, the surgeon will also likely remove some lymph nodes under the arms in order to check to see if the cancer has spread from the breast.

Radiation therapy Triple Negative Breast Cancer Treatment. Usually given after surgery, radiation therapy is the use of high-energy X-rays to kill the breast cancer cells. It can be given externally, meaning the radiation stems from a large machine, or internally, where the radiation is placed in a small tube and inserted into the breast through a tiny incision.

Chemotherapy Triple Negative Breast Cancer Treatment. Chemotherapy has been shown to be the most effective triple-negative breast cancer treatment option because of the way it works in killing the rapidly dividing cancer cells. The most common chemotherapy regimen used includes a combination of anthracyclin such as doxorubicin and cyclophosphamide, which is commonly referred to as ‘AC.’ Some patients also are treated with a third drug—either fluorouracil (5-FU), Taxol (paclitaxel) or Taxotere (docetaxel) along with AC chemotherapy. Other patients may be treated with another anthracyclin, epirubicin, instead of the doxorubicin, which is then called an ‘EC’ regimen. Also promising results are obtained with a treatment with monoclonal antibody against VEGF-A (bevacizumab (Avastin)), and chemotherapy drug paclitaxel (Taxol). Cis-platin compounds are also being tested, usually in combination with some chemotherapy such as anthracyclin.

As used herein, the terms treatment, treat, or treating refers to a method of reducing the effects of a disease or condition or symptom of the disease or condition. Thus, in the disclosed method, treatment can refer to a 10%, 20%, 30%, 40%, 50%, 60%, 70%, 80%, 90%, or 100% reduction in the severity of an established disease or condition or symptom of the disease or condition. For example, a method of treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100% or any percent reduction between 10 and 100% as compared to native or control levels. It is understood that treatment does not necessarily refer to a cure or complete ablation of the disease, condition, or symptoms of the disease or condition.

For the purpose of the present invention, the reference level of expression of a biomarker is the median expression of the biomarker from a group of triple negative breast cancer cells. Preferably at least 20 different triple negative breast cancer tissues are used to obtain the reference level of expression. More preferably at least 30 different triple negative breast cancer tissues are used, more preferably at least 40 different triple negative breast cancer tissues are used, even more preferably at least 50 different triple negative breast cancer tissues are used, more preferably at least 60 different triple negative breast cancer tissues are used. It is to be understood the more different breast cancer tissues are used the more reliable the reference expression can be determined. There may be several statistical analyses to determine the median expression level of a biomarker. Suitably a Z-score is used to determine the median expression of a biomarker from a group of breast cancer tissues.

Up-regulated expression is defined as significantly more than median. There exist several statistical analyses to determine whether an expression is significantly more than the median. The level of significance may be 10% (0.1), more preferably, 5% (0.05), even more preferably 1% (0.01), even more preferably 0.5% (0.005), and most preferably 0.1% (0.001).

Down-regulated expression is defined as significantly less than median. There exist several statistical analyses to determine whether an expression is significantly less than the median. The level of significance may be 10% (0.1), more preferably, 5% (0.05), even more preferably 1% (0.01), even more preferably 0.5% (0.005), and most preferably 0.1% (0.001).

Expression levels may determined by any assays known to a skilled person. Examples are microarrays, DNA, RNA and protein, chemoluminescense assays, fluorescence assays, mass spectrometry, affinity chromotograpy, blotting, electrophoresis, histology, linkers, protein expression chip, probes. Preferred are multiplex systems that can measure more than one protein, peptide or gene at one time. A suitable multiplex system is multiple reaction monitoring (MRM), which is a quantitative MS-based approach. Mass spectrometry is a suitable means of determining the level of expression of peptides and proteins.

DNA microarrays allow for the parallel measurement of thousands of genes on the level of mRNA. Protein microarrays increase the throughput of proteomic research and increase the quantity of data points in small biological samples on the protein level. Microarrays of antibodies can simultaneously measure the concentration of a multitude of target proteins in a very short period of time. Protein expression can be quantified using either protein tags or fluorescently or chemo luminescent labelled antibodies. Mass spectrometry can be used both quantitatively and qualitatively.

### DETAILED DESCRIPTION

The present invention relates to a method for determining a prognosis for a patient with triple negative breast cancer. For the method the level of expression is determined of at least one biomarker selected from the group comprising AP1G1 and/or CAPZB and or from the group comprising MTHFD1, CTNNA1, STX12, and/or AP1M1 and/or from the group comprising

CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, K1AA0174, HLA-C, UBE2Q1, PSMB9, AGL, GOSR1, PTK2, SMC4, HAPLN1, SKIV2L, and/or GSTM1, in a biological sample from said patient. Triple negative breast cancer (TNBC) cells test negative for estrogen receptors (ER-), progesterone receptors (PR-), and HER2 (HER2-). Testing negative for all three means the cancer is triple-negative. These negative results mean that the growth of the cancer is not supported by the hormones estrogen and progesterone, nor by the presence of too many HER2 receptors. Therefore, triple-negative breast cancer does not respond to hormonal therapy (such as tamoxifen or aromatase inhibitors) or therapies that target HER2 receptors, such as Herceptin (chemical name: trastuzumab). However, other non-targeted (chemotherapy) medicines can be used to treat triple-negative breast cancer. The main chemotherapy treatment for triple negative breast cancer is usually a combination of chemotherapy drugs. The combination often include an anthracycline, such as daunorubicin, doxorubicin or epirubicin. In a randomised phase 3 trial, the monoclonal antibody against VEGF-A (bevacizumab (Avastin)) and chemotherapy drug paclitaxel (Taxol) appeared to control advanced breast cancer for a time in some women with triple negative breast cancer. Researchers are currently studying various types of biological therapy including olaparib, a PARP-1 inhibitor.

About 10-20% of breast cancers are found to be triple-negative. Triple-negative breast cancer tends to be more aggressive than other types of breast cancer. Studies have shown that triple-negative breast cancer is more likely to spread beyond the breast and more likely to recur (come back) after treatment. These risks appear to be greatest in the first few years after treatment. For example, a study of more than 1,600 women in Canada published in 2007 found that women with triple-negative breast cancer were at higher risk of having the cancer recur outside the breast—but only for the first 3 years. Other studies have reached similar conclusions. As years go by, the risks of the triple-negative breast cancer recurring become similar to those risk levels for other types of breast cancer. Five-year survival rates also tend to be lower for triple-negative breast cancer. A 2007 study of more than 50,000 women with all stages of breast cancer found that 77% of women with triple-negative breast cancer survived at least 5 years, versus 93% of women with other types of breast cancer. Another study of more than 1,600 women published in 2007 found that women with triple-negative breast cancer had a higher risk of death within 5 years of diagnosis, but not after that time period.

Triple negative breast cancer also tends to be higher grade than other types of breast cancer. The higher the grade, the less the cancer cells resemble normal, healthy breast cells in their appearance and growth patterns. On a scale of 1 to 3, triple-negative breast cancer often is grade 3.

Usually triple negative breast cancer is a cell type called “basal-like.” “Basal-like” means that the breast cancer cells express cytokeratines such as CK5 and CK17, which are also expressed in healthy breast tissue in basal cells that line the breast ducts. This is a new subtype of breast cancer that researchers have identified using gene analysis technology. Like other types of breast cancer, basal-like cancers can be linked to family history, or they can happen without any apparent family link. Basal-like cancers tend to be more aggressive, higher grade cancers—just like triple-negative breast cancers. Most triple-negative breast cancers are of the basal-like intrinsic subtype. Some TNBC over expresses epidermal growth factor receptor (EGFR). Some TNBC over expresses transmembrane glycoprotein NMB (GPNMB).

On histologic examination triple negative breast tumors mostly fall into the categories secretory carcinoma or adenoid cystic types (both considered less aggressive), medullary cancers and grade 3 invasive ductal carcinomas with no specific subtype, and highly aggressive metastatic cancers. Medullary TNBC in younger women are frequently BRCA1-related.

A biomarker may be a protein, nucleic acid encoding for a protein, peptides of a protein, fragments of protein, or mutants thereof, and or metabolites, or lipids. Fragments or mutants preferably have at least 70% sequence identity to the biomarker as disclosed herein. More preferably at least 75% sequence identity, more preferably at least 80% sequence identity, more preferably at least 85% sequence identity, more preferably at least 90% sequence identity, more preferably at least 92% sequence identity, more preferably at least 94% sequence identity, more preferably at least 95% sequence identity, more preferably at least 97% sequence identity, more preferably at least 99% sequence identity. Preferred biomarkers are proteins, peptides, or nucleic acids coding for a peptide or protein, or fragments and/or mutants thereof. Most preferred biomarkers are peptides and/or proteins and/or mutants and/or fragments of these peptides and/or proteins.

In a preferred method of the present invention and embodiments thereof the biological sample is selected from the group consisting of tumor cells, tissue, blood, serum, plasma, urine, circulating tumour cells, nipple aspirate fluid, cerebrospinal fluid, sputum, aerosols, breast tissue, and/or thrombocytes.

The level of expression of the biomarker may be determined by any method known to a skilled person and will depend on the nature of the biomarker. Preferably the expression of the biomarker is determined by a technique selected from the group consisting of mass spectrometry, DNA array, immunohistochemistry, antibodies based assay, probe-based assay. Preferably the expression is determined by mass spectrometry. In a preferred method of the present invention and/or embodiments thereof the technique is a multiplex technique allowing for more than one biomarker to be analysed at the same time.

It is to be understood that the patient is already diagnosed with triple-negative breast cancer. Any known technique may be used to diagnose a person with triple negative breast cancer. A person is diagnosed triple negative breast cancer when the breast cancer tissue does not express ER, PR and HER2.

In a preferred method according to the invention and/or embodiments thereof it is further established whether the expression of said biomarker is up-regulated or down-regulated. Up or down-regulation may be compared to a reference level of said biomarker. A preferred reference level is the median expression of the biomarker in a group of triple negative breast cancer tissues. Preferably at least 20 different triple negative breast cancer tissues are used to obtain the reference level. More preferably at least 30 different triple negative breast cancer tissues are used, more preferably at least 40 different triple negative breast cancer tissues are used, even more preferably at least 50 different triple negative breast cancer tissues are used, more preferably at least 60 different triple negative breast cancer tissues are used. It is to be understood the more different breast cancer tissues are used the more reliable the reference level may be determined. There may be several statistical analyses to determine the median expression level of a biomarker. Suitably a Z-score is used to determine the median expression of a biomarker from a group of breast cancer tissues.

Up-regulated expression is defined as significantly more than median. There exist several statistical analyses to determine whether an expression is significantly more than the median. The level of significance may be 10% (0.1), more preferably, 5% (0.05), even more preferably 1% (0.01), even more preferably 0.5% (0.005), and most preferably 0.1% (0.001).

Down-regulated expression is defined as significantly less than median. There exist several statistical analyses to determine whether an expression is significantly less than the median. The level of significance may be 10% (0.1), more preferably, 5% (0.05), even more preferably 1% (0.01), even more preferably 0.5% (0.005), and most preferably 0.1% (0.001).

In a preferred method according to the invention and/or embodiments thereof the level of expression of is MTHFD1 is upregulated and correlates with poor prognosis of said patient. In a preferred method according to the invention and/or embodiments thereof the level of expression of at least one biomarker selected from the group consisting of PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, UBE2Q1, SMC4, and/or HAPLN1, is up-regulated and correlates with poor prognosis of said patient.

In another preferred method according to the invention and/or embodiments thereof and/or the level expression of at least one biomarker selected from the group consisting of AP1G1, CAPZB, CTNNA1, STX12, and/or AP1M1 is down-regulated in said sample and correlates with poor prognosis of said patient.

In another preferred method according to the invention and/or embodiments thereof and/or the level expression of at least one biomarker selected from the group consisting of

CMPK1, PRKACA, PRKACB, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM is down-regulated in said sample and correlates with poor prognosis of said patient. Poor prognosis is the development of distant metastasis within 5 year after diagnosis. This poor prognosis is even after treatment is given.

In a preferred method according to the invention and/or embodiments thereof the level of expression of at least one biomarker selected from the group consisting of GPRC5A, LPCAT1, ACTBL2, SIGMAR1, CPT1A, SFXN2, RBBP7, BAZ1B, PPOX, FLAD1, MIF, FDPS, C8orf55, KTN1, GTPBP4, is up-regulated and correlates with poor prognosis of said patient.

In a preferred method according to the invention and/or embodiments thereof the level of expression of at least one biomarker selected from the group consisting of MIF, FDPS, ACTBL2, KTN1, C8orf55, GTPBP4, RBBP7, FLAD1, PPOX, is up-regulated and correlates with poor prognosis of said patient.

In a preferred method according to the invention and/or embodiments thereof the level of expression of at least one biomarker selected from the group consisting of GPRC5A, LPCAT1, ACTBL2, SIGMAR1, CPT1A, PPOX, FLAD1, MIF, FDPS, is up-regulated and correlates with poor prognosis of said patient.

In a preferred method according to the invention and/or embodiments thereof the level of expression of at least one biomarker selected from the group consisting of GPRC5A, LPCAT1, ACTBL2, PPOX, FLAD1, is up-regulated and correlates with poor prognosis of said patient.

In a preferred method according to the invention and/or embodiments thereof the level of expression of at least one biomarker selected from the group consisting of ACTBL2, PPOX, FLAD1, is up-regulated and correlates with poor prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A is down-regulated in said sample and correlates with poor prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A is down-regulated in said sample and correlates with poor prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A is down-regulated in said sample and correlates with poor prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A is down-regulated in said sample and correlates with poor prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A is down-regulated in said sample and correlates with poor prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A is down-regulated in said sample and correlates with poor prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A is down-regulated in said sample and correlates with poor prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A is down-regulated in said sample and correlates with poor prognosis of said patient. Preferably the biomarker is not FTH1, and/or TF and/or YWHAQ.

In another preferred method according to the invention and/or embodiments thereof and/or the level expression of at least one biomarker selected from the group consisting of CMPK1, PRKACA, EML4, GANAB, PRKAR1A, PSME2, STX5, MDH1, FTH1, OTUB1, MGP, TF is down-regulated in said sample and correlates with poor prognosis of said patient.

In another preferred method according to the invention and/or embodiments thereof and/or the level expression of at least one biomarker selected from the group consisting of CMPK1, PRKACA, EML4, GANAB, PRKAR1A, PSME2, STX5, MDH1, OTUB1, MGP, TF is down-regulated in said sample and correlates with poor prognosis of said patient.

In another preferred method according to the invention and/or embodiments thereof and/or the level expression of at least one biomarker selected from the group consisting of CMPK1, PRKACA, EML4, GANAB, PRKAR1A, PSME2, STX5, MDH1, FTH1, OTUB1, MGP is down-regulated in said sample and correlates with poor prognosis of said patient.

In another preferred method according to the invention and/or embodiments thereof and/or the level expression of at least one biomarker selected from the group consisting of CMPK1, PRKACA, EML4, GANAB, PRKAR1A, PSME2, STX5, MDH1, OTUB1, MGP, is down-regulated in said sample and correlates with poor prognosis of said patient.

In another preferred method according to the invention and/or embodiments thereof and/or the level expression of at least one biomarker selected from the group consisting of CMPK1, APIP, STX5, AASDHPPT, MARCKSL1, PRKACA, PRKACB, EML4, GANAB, RAB1A is down-regulated in said sample and correlates with poor prognosis of said patient.

In another preferred method according to the invention and/or embodiments thereof and/or the level expression of at least one biomarker selected from the group consisting of CMPK1, PRKACA, EML4, GANAB, PRKAR1A is down-regulated in said sample and correlates with poor prognosis of said patient.

Poor prognosis is the development of distant metastasis within 5 year after diagnosis. This poor prognosis is even after treatment is given.

In a preferred method according to the invention and/or embodiments thereof the level expression of at least one biomarker selected from the group consisting of PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, UBE2Q1, SMC4, and/or HAPLN1 is down-regulated in said patient and correlates with good prognosis. In a preferred method according to the invention and/or embodiments thereof the level expression of at least one biomarker selected from the group consisting of CMPK1, PRKACA; PRKACB, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM is up-regulated in said sample correlates with good prognosis.

In a preferred method according to the invention and/or embodiments thereof the level of expression of at least one biomarker selected from the group consisting of GPRC5A, LPCAT1, ACTBL2, SIGMAR1, CPT1A, SFXN2, RBBP7, BAZ1B, PPOX, FLAD1, MIF, FDPS, C8orf55, KTN1, GTPBP4, is down-regulated and correlates with good prognosis of said patient.

In a preferred method according to the invention and/or embodiments thereof the level of expression of at least one biomarker selected from the group consisting of MIF, FDPS, ACTBL2, KTN1, C8orf55, GTPBP4, RBBP7, FLAD1, PPOX, is down-regulated and correlates with good prognosis of said patient.

In a preferred method according to the invention and/or embodiments thereof the level of expression of at least one biomarker selected from the group consisting of GPRC5A, LPCAT1, ACTBL2, SIGMAR1, CPT1A, PPOX, FLAD1, MIF, FDPS, is down-regulated and correlates with good prognosis of said patient.

In a preferred method according to the invention and/or embodiments thereof the level of expression of at least one biomarker selected from the group consisting of GPRC5A, LPCAT1, ACTBL2, PPOX, FLAD1, is down-regulated and correlates with good prognosis of said patient.

In a preferred method according to the invention and/or embodiments thereof the level of expression of at least one biomarker selected from the group consisting of ACTBL2, PPOX, FLAD1, is down-regulated and correlates with good prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A is up-regulated in said sample and correlates with good prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A is up-regulated in said sample and correlates with good prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A is up-regulated in said sample and correlates with good prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A is up-regulated in said sample and correlates with good prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A is up-regulated in said sample and correlates with good prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A is up-regulated in said sample and correlates with good prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A is up-regulated in said sample and correlates with good prognosis of said patient.

In another preferred method according to the invention and/or embodiments thereof and/or the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A is up-regulated in said sample and correlates with good prognosis of said patient. Preferably the biomarker is not FTH1, and/or TF and/or YWHAQ.

In another preferred method according to the invention and/or embodiments thereof and/or the level expression of at least one biomarker selected from the group consisting of CMPK1, PRKACA, EML4, GANAB, PRKAR1A, PSME2, STX5, MDH1, FTH1, OTUB1, MGP, TF is up-regulated in said sample and correlates with good prognosis of said patient.

In another preferred method according to the invention and/or embodiments thereof and/or the level expression of at least one biomarker selected from the group consisting of CMPK1, PRKACA, EML4, GANAB, PRKAR1A, PSME2, STX5, MDH1, OTUB1, MGP, TF is up-regulated in said sample and correlates with good prognosis of said patient.

In another preferred method according to the invention and/or embodiments thereof and/or the level expression of at least one biomarker selected from the group consisting of CMPK1, PRKACA, EML4, GANAB, PRKAR1A, PSME2, STX5, MDH1, FTH1, OTUB1, MGP is up-regulated in said sample and correlates with good prognosis of said patient.

In another preferred method according to the invention and/or embodiments thereof and/or the level expression of at least one biomarker selected from the group consisting of CMPK1, PRKACA, EML4, GANAB, PRKAR1A, PSME2, STX5, MDH1, OTUB1, MGP, is up-regulated in said sample and correlates with good prognosis of said patient.

In another preferred method according to the invention and/or embodiments thereof and/or the level expression of at least one biomarker selected from the group consisting of CMPK1, APIP, STX5, AASDHPPT, MARCKSL1, PRKACA, PRKACB, EML4, GANAB, RAB1A is up-regulated in said sample and correlates with good prognosis of said patient.

In another preferred method according to the invention and/or embodiments thereof and/or the level expression of at least one biomarker selected from the group consisting of CMPK1, PRKACA, EML4, GANAB, PRKAR1A is up-regulated in said sample and correlates with good prognosis of said patient.

Good prognosis is being metastasis free for at least 5 years. The good prognosis is expected when treatment is given. The advantage of the present invention is that patients with good prognosis may be selected to receive treatment. Treatment of triple negative breast cancer often comprises the use of chemotherapy that may have severe side-effects. Patients with poor prognosis may choose not to undergo treatment such as X-ray radiation and/or chemotherapy to avoid the side-effects of these treatments. In addition, treatment protocols for triple negative breast cancer may be based on the markers and methods as disclosed in the present invention.

The present invention and/or embodiments thereof is also related to the use of a protein or a nucleic acid coding for a protein selected from group consisting of CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, K1AA0174, HLA-C, UBE2Q1, PSMB9, AGL, GOSR1, PTK2, SMC4, HAPLN1, SKIV2L, and/or GSTM1 as biomarker to determine prognosis in triple negative breast cancer.

In a preferred use of the present invention and/or embodiments thereof the prognosis is poor or good and may indicate an increased or diminished survival chance.

The present invention is also related to a method of determining effectiveness of treatment for a patient with triple negative breast cancer comprising determining at a first time point the level of expression at least one biomarker selected from the group comprising CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1 in a biological sample from said patient and then determining at a second time point the level of expression at least one biomarker selected from the group comprising CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDCl₂₃, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, K1AA0174, HLA-C, UBE2Q1, PSMB9, AGL, GOSR1, PTK2, SMC4, HAPLN1, SKIV2L, and/or GSTM1 in a biological sample from said patient.

In a preferred method of the present invention and/or embodiments thereof the biomarker at the first and second time point are the same biomarker. Preferably the difference in expression level between the first an second time point is determined. Preferably the second time point is after treatment is given. In a preferred method of the present invention and/or embodiments thereof the level of expression of at least one biomarker between the first and second time point does not show a significant different or the difference is small. A small difference is less than 0.3 log 2 fold between the level of expression of the first time point and the second time point. No significant difference or a small difference is indicative of the effectiveness of the treatment given being low. The level of significance is preferably 10%, more preferably 5%, more preferably 1%, more preferably 0.5% and most preferably 0.1%.

In a preferred method of the present invention and/or embodiments thereof the level of expression of at least one biomarker selected from the group consisting of PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, UBE2Q1, SMC4, and/or HAPLN1 is higher at the second time point than at the first time point and/or the level expression of at least one biomarker selected from the group consisting of CMPK1, PRKACA; PRKACB, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM is lower at the second time point than at the first time point and is indicative of the treatment being low effective.

A low effective treatment does not significantly change the prognosis of triple negative breast cancer and/or does not changes the survival rate of a patient.

In a preferred method of the present invention and/or embodiments thereof the level of expression of at least one biomarker selected from the group consisting of GPRC5A, LPCAT1, ACTBL2, SIGMAR1, CPT1A, SFXN2, RBBP7, BAZ1B, PPOX, FLAD1, MIF, FDPS, C8orf55, KTN1, GTPBP4 is higher at the second time point than at the first time point and is indicative of the treatment being low effective.

In a preferred method of the present invention and/or embodiments thereof the level of expression of at least one biomarker selected from the group consisting of MIF, FDPS, ACTBL2, KTN1, C8orf55, GTPBP4, RBBP7, FLAD1, PPOX is higher at the second time point than at the first time point and is indicative of the treatment being low effective.

In a preferred method of the present invention and/or embodiments thereof the level of expression of at least one biomarker selected from the group consisting of GPRC5A, LPCAT1, ACTBL2, SIGMAR1, CPT1A, PPOX, FLAD1, MIF, FDPS is higher at the second time point than at the first time point and is indicative of the treatment being low effective.

In a preferred method of the present invention and/or embodiments thereof the level of expression of at least one biomarker selected from the group consisting of GPRC5A, LPCAT1, ACTBL2, PPOX, FLAD1 is higher at the second time point than at the first time point and is indicative of the treatment being low effective.

In a preferred method of the present invention and/or embodiments thereof the level of expression of at least one biomarker selected from the group consisting of ACTBL2, PPOX, FLAD1 is higher at the second time point than at the first time point and is indicative of the treatment being low effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A is lower at the second time point than at the first time point and is indicative of the treatment being low effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A is lower at the second time point than at the first time point and is indicative of the treatment being low effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A is lower at the second time point than at the first time point and is indicative of the treatment being low effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A is lower at the second time point than at the first time point and is indicative of the treatment being low effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A is lower at the second time point than at the first time point and is indicative of the treatment being low effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A is lower at the second time point than at the first time point and is indicative of the treatment being low effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A is lower at the second time point than at the first time point and is indicative of the treatment being low effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A is than at the first time point and is indicative of the treatment being low effective. Preferably the biomarker is not FTH1, and/or TF and/or YWHAQ.

In a preferred method of the present invention and/or embodiments thereof the level of expression of at least one biomarker selected from the group consisting of CMPK1, PRKACA, EML4, GANAB, PRKAR1A, PSME2, STX5, MDH1, FTH1, OTUB1, MGP, TF is lower at the second time point than at the first time point and is indicative of the treatment being low effective.

In a preferred method of the present invention and/or embodiments thereof the level of expression of at least one biomarker selected from the group consisting of CMPK1, PRKACA, EML4, GANAB, PRKAR1A, PSME2, STX5, MDH1, OTUB1, MGP, TF is lower at the second time point than at the first time point and is indicative of the treatment being low effective.

In a preferred method of the present invention and/or embodiments thereof the level of expression of at least one biomarker selected from the group consisting of CMPK1, PRKACA, EML4, GANAB, PRKAR1A, PSME2, STX5, MDH1, FTH1, OTUB1, MGP is lower at the second time point than at the first time point and is indicative of the treatment being low effective.

In a preferred method of the present invention and/or embodiments thereof the level of expression of at least one biomarker selected from the group consisting of CMPK1, PRKACA, EML4, GANAB, PRKAR1A, PSME2, STX5, MDH1, OTUB1, MGP, is lower at the second time point than at the first time point and is indicative of the treatment being low effective.

In a preferred method of the present invention and/or embodiments thereof the level of expression of at least one biomarker selected from the group consisting of CMPK1, APIP, STX5, AASDHPPT, MARCKSL1, PRKACA, PRKACB, EML4, GANAB, RAB1A is lower at the second time point than at the first time point and is indicative of the treatment being low effective.

In a preferred method of the present invention and/or embodiments thereof the level of expression of at least one biomarker selected from the group consisting of CMPK1, PRKACA, EML4, GANAB, PRKAR1A is lower at the second time point than at the first time point and is indicative of the treatment being low effective.

In a preferred method of the present invention and/or embodiments thereof the level of expression of at least one biomarker selected from the group consisting PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, UBE2Q1, SMC4, and/or HAPLN1 is lower at the second time point than at the first time point and/or the level expression of at least one biomarker selected from the group consisting of CMPK1, PRKACA; PRKACB, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM is higher at the second time point than at the first time point, is indicative the effectiveness of the treatment given being high. An effective treatment significantly chances the prognosis of the patient from poor to good and/or significantly increases the survival rate.

In a preferred method of the present invention and/or embodiments thereof the level of expression of at least one biomarker selected from the group consisting GPRC5A, LPCAT1, ACTBL2, SIGMAR1, CPT1A, SFXN2, RBBP7, BAZ1B, PPOX, FLAD1, MIF, FDPS, C8orf55, KTN1, GTPBP4, is lower at the second time point than at the first time point is and indicative the effectiveness of the treatment given being high.

In a preferred method of the present invention and/or embodiments thereof the level of expression of at least one biomarker selected from the group consisting MIF, FDPS, ACTBL2, KTN1, C8orf55, GTPBP4, RBBP7, FLAD1, PPOX, is lower at the second time point than at the first time point is and indicative the effectiveness of the treatment given being high.

In a preferred method of the present invention and/or embodiments thereof the level of expression of at least one biomarker selected from the group consisting GPRC5A, LPCAT1, ACTBL2, SIGMAR1, CPT1A, PPOX, FLAD1, MIF, FDPS, is lower at the second time point than at the first time point is and indicative the effectiveness of the treatment given being high.

In a preferred method of the present invention and/or embodiments thereof the level of expression of at least one biomarker selected from the group consisting GPRC5A, LPCAT1, ACTBL2, PPOX, FLAD1, is lower at the second time point than at the first time point is and indicative the effectiveness of the treatment given being high.

In a preferred method of the present invention and/or embodiments thereof the level of expression of at least one biomarker selected from the group consisting ACTBL2, PPOX, FLAD1, is lower at the second time point than at the first time point is and indicative the effectiveness of the treatment given being high.

In a preferred method of the present invention and/or embodiments thereof the level of expression of at least one biomarker selected from the group consisting CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A is higher at the second time point than at the first time point and is indicative the effectiveness of the treatment given being high.

In a preferred method of the present invention and/or embodiments thereof the level of expression of at least one biomarker selected from the group consisting CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A is higher at the second time point than at the first time point and is indicative the effectiveness of the treatment given being high.

In a preferred method of the present invention and/or embodiments thereof the level of expression of at least one biomarker selected from the group consisting CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A is higher at the second time point than at the first time point and is indicative the effectiveness of the treatment given being high.

In a preferred method of the present invention and/or embodiments thereof the level of expression of at least one biomarker selected from the group consisting CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A is higher at the second time point than at the first time point and is indicative the effectiveness of the treatment given being high.

In a preferred method of the present invention and/or embodiments thereof the level of expression of at least one biomarker selected from the group consisting CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A is higher at the second time point than at the first time point and is indicative the effectiveness of the treatment given being high.

In a preferred method of the present invention and/or embodiments thereof the level of expression of at least one biomarker selected from the group consisting CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A is higher at the second time point than at the first time point and is indicative the effectiveness of the treatment given being high.

In a preferred method of the present invention and/or embodiments thereof the level of expression of at least one biomarker selected from the group consisting CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A is higher at the second time point than at the first time point and is indicative the effectiveness of the treatment given being high.

In a preferred method of the present invention and/or embodiments thereof the level of expression of at least one biomarker selected from the group consisting CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A is higher at the second time point than at the first time point and is indicative the effectiveness of the treatment given being high. Preferably the biomarker is not FTH1, and/or TF and/or YWHAQ.

In a preferred method of the present invention and/or embodiments thereof the level of expression of at least one biomarker selected from the group consisting CMPK1, PRKACA, EML4, GANAB, PRKAR1A, PSME2, STX5, MDH1, FTH1, OTUB1, MGP, TF is higher at the second time point than at the first time point and is indicative the effectiveness of the treatment given being high.

In a preferred method of the present invention and/or embodiments thereof the level of expression of at least one biomarker selected from the group consisting CMPK1, PRKACA, EML4, GANAB, PRKAR1A, PSME2, STX5, MDH1, OTUB1, MGP, TF is higher at the second time point than at the first time point and is indicative the effectiveness of the treatment given being high.

In a preferred method of the present invention and/or embodiments thereof the level of expression of at least one biomarker selected from the group consisting CMPK1, PRKACA, EML4, GANAB, PRKAR1A, PSME2, STX5, MDH1, FTH1, OTUB1, MGP is higher at the second time point than at the first time point and is indicative the effectiveness of the treatment given being high.

In a preferred method of the present invention and/or embodiments thereof the level of expression of at least one biomarker selected from the group consisting CMPK1, PRKACA, EML4, GANAB, PRKAR1A, PSME2, STX5, MDH1, OTUB1, MGP, is higher at the second time point than at the first time point and is indicative the effectiveness of the treatment given being high.

In a preferred method of the present invention and/or embodiments thereof the level of expression of at least one biomarker selected from the group consisting CMPK1, APIP, STX5, AASDHPPT, MARCKSL1, PRKACA, PRKACB, EML4, GANAB, RAB1A is higher at the second time point than at the first time point and is indicative the effectiveness of the treatment given being high.

In a preferred method of the present invention and/or embodiments thereof the level of expression of at least one biomarker selected from the group consisting CMPK1, PRKACA, EML4, GANAB, PRKAR1A is higher at the second time point than at the first time point and is indicative the effectiveness of the treatment given being high.

The present invention also relates to a method of determining treatment for a patient with triple negative breast cancer comprising determining a level of expression of at least one biomarker selected from the group comprising CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDCl₂₃, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, K1AA0174, HLA-C, UBE2Q1, PSMB9, AGL, GOSR1, PTK2, SMC4, HAPLN1, SKIV2L, and/or GSTM1 in a biological sample from said patient.

In a preferred method of the present invention and/or embodiments thereof at a first and at a second time point the expression level of the biomarker is determined. Preferably the biomarker at the first and second time point are the same. Preferably the second time point is after treatment is given. In a preferred method of the present invention and/or embodiments thereof the level expression of at least one biomarker selected from the group consisting of PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, UBE2Q1, SMC4, and/or HAPLN1 is down-regulated and/or the level expression of at least one biomarker selected from the group consisting of CMPK1, PRKACA; PRKACB, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM is up-regulated in said sample and is indicative of the treatment being effective.

In a preferred method of the present invention and/or embodiments thereof the level expression of at least one biomarker selected from the group consisting of GPRC5A, LPCAT1, ACTBL2, SIGMAR1, CPT1A, SFXN2, RBBP7, BAZ1B, PPOX, FLAD1, MIF, FDPS, C8orf55, KTN1, GTPBP4 is down-regulated and is indicative of a treatment being effective.

In a preferred method of the present invention and/or embodiments thereof the level expression of at least one biomarker selected from the group consisting of MIF, FDPS, ACTBL2, KTN1, C8orf55, GTPBP4, RBBP7, FLAD1, PPOX is down-regulated and is indicative of a treatment being effective.

In a preferred method of the present invention and/or embodiments thereof the level expression of at least one biomarker selected from the group consisting of GPRC5A, LPCAT1, ACTBL2, SIGMAR1, CPT1A, PPOX, FLAD1, MIF, FDPS is down-regulated and is indicative of a treatment being effective.

In a preferred method of the present invention and/or embodiments thereof the level expression of at least one biomarker selected from the group consisting of GPRC5A, LPCAT1, ACTBL2, PPOX, FLAD1 is down-regulated and is indicative of a treatment being effective.

In a preferred method of the present invention and/or embodiments thereof the level expression of at least one biomarker selected from the group consisting of ACTBL2, PPOX, FLAD1 is down-regulated and is indicative of a treatment being effective.

In a preferred method of the present invention and/or embodiments thereof the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A is up-regulated in said sample and is indicative of the treatment being effective.

In a preferred method of the present invention and/or embodiments thereof the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A is up-regulated in said sample and is indicative of the treatment being effective.

In a preferred method of the present invention and/or embodiments thereof the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A is up-regulated in said sample and is indicative of the treatment being effective.

In a preferred method of the present invention and/or embodiments thereof the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A is up-regulated in said sample and is indicative of the treatment being effective.

In a preferred method of the present invention and/or embodiments thereof the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A is up-regulated in said sample and is indicative of the treatment being effective.

In a preferred method of the present invention and/or embodiments thereof the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A is up-regulated in said sample and is indicative of the treatment being effective.

In a preferred method of the present invention and/or embodiments thereof the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A.

In a preferred method of the present invention and/or embodiments thereof the level expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A is. Preferably the biomarker is not FTH1, and/or TF and/or YWHAQ.

In a preferred method of the present invention and/or embodiments thereof the level expression of at least one biomarker selected from the group consisting of CMPK1, PRKACA, EML4, GANAB, PRKAR1A, PSME2, STX5, MDH1, FTH1, OTUB1, MGP, TF is up-regulated in said sample and is indicative of the treatment being effective.

In a preferred method of the present invention and/or embodiments thereof the level expression of at least one biomarker selected from the group consisting of CMPK1, PRKACA, EML4, GANAB, PRKAR1A, PSME2, STX5, MDH1, OTUB1, MGP, TF is up-regulated in said sample and is indicative of the treatment being effective.

In a preferred method of the present invention and/or embodiments thereof the level expression of at least one biomarker selected from the group consisting of CMPK1, PRKACA, EML4, GANAB, PRKAR1A, PSME2, STX5, MDH1, FTH1, OTUB1, MGP is up-regulated in said sample and is indicative of the treatment being effective.

In a preferred method of the present invention and/or embodiments thereof the level expression of at least one biomarker selected from the group consisting of CMPK1, PRKACA, EML4, GANAB, PRKAR1A, PSME2, STX5, MDH1, OTUB1, MGP, is up-regulated in said sample and is indicative of the treatment being effective.

In a preferred method of the present invention and/or embodiments thereof the level expression of at least one biomarker selected from the group consisting of CMPK1, APIP, STX5, AASDHPPT, MARCKSL1, PRKACA, PRKACB, EML4, GANAB, RAB1A is up-regulated in said sample and is indicative of the treatment being effective.

In a preferred method of the present invention and/or embodiments thereof the level expression of at least one biomarker selected from the group consisting of CMPK1, PRKACA, EML4, GANAB, PRKAR1A is up-regulated in said sample and is indicative of the treatment being effective.

In a preferred method of the present invention and/or embodiments thereof the level of expression of at least one biomarker selected from the group consisting of PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, UBE2Q1, SMC4, and/or HAPLN1 is up-regulated and/or the level expression of at least one biomarker selected from the group consisting of CMPK1, PRKACA; PRKACB, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM is down-regulated in said sample and is indicative of the treatment not being effective.

In a preferred method of the present invention and/or embodiments thereof the level of expression of at least one biomarker selected from the group consisting of GPRC5A, LPCAT1, ACTBL2, SIGMAR1, CPT1A, SFXN2, RBBP7, BAZ1B, PPOX, FLAD1, MIF, FDPS, C8orf55, KTN1, GTPBP4 is up-regulated and is indicative of the treatment not being effective.

In a preferred method of the present invention and/or embodiments thereof the level of expression of at least one biomarker selected from the group consisting of MIF, FDPS, ACTBL2, KTN1, C8orf55, GTPBP4, RBBP7, FLAD1, PPOX is up-regulated and is indicative of the treatment not being effective.

In a preferred method of the present invention and/or embodiments thereof the level of expression of at least one biomarker selected from the group consisting of GPRC5A, LPCAT1, ACTBL2, SIGMAR1, CPT1A, PPOX, FLAD1, MIF, FDPS is up-regulated and is indicative of the treatment not being effective.

In a preferred method of the present invention and/or embodiments thereof the level of expression of at least one biomarker selected from the group consisting of GPRC5A, LPCAT1, ACTBL2, PPOX, FLAD1 is up-regulated and is indicative of the treatment not being effective.

In a preferred method of the present invention and/or embodiments thereof the level of expression of at least one biomarker selected from the group consisting of ACTBL2, PPOX, FLAD1 is up-regulated and is indicative of the treatment not being effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A is down-regulated in said sample and is indicative of the treatment not being effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A is down-regulated in said sample and is indicative of the treatment not being effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A is down-regulated in said sample and is indicative of the treatment not being effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A is down-regulated in said sample and is indicative of the treatment not being effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A is down-regulated in said sample and is indicative of the treatment not being effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A is down-regulated in said sample and is indicative of the treatment not being effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A is down-regulated in said sample and is indicative of the treatment not being effective.

In a preferred method of the present invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A is. Preferably the biomarker is not FTH1, and/or TF and/or YWHAQ.

In a preferred method of the present invention and/or embodiments thereof the level of expression of at least one biomarker selected from the group consisting of CMPK1, PRKACA, EML4, GANAB, PRKAR1A, PSME2, STX5, MDH1, FTH1, OTUB1, MGP, TF is down-regulated in said sample and is indicative of the treatment not being effective.

In a preferred method of the present invention and/or embodiments thereof the level of expression of at least one biomarker selected from the group consisting of CMPK1, PRKACA, EML4, GANAB, PRKAR1A, PSME2, STX5, MDH1, OTUB1, MGP, TF is down-regulated in said sample and is indicative of the treatment not being effective.

In a preferred method of the present invention and/or embodiments thereof the level of expression of at least one biomarker selected from the group consisting of CMPK1, PRKACA, EML4, GANAB, PRKAR1A, PSME2, STX5, MDH1, FTH1, OTUB1, MGP is down-regulated in said sample and is indicative of the treatment not being effective.

In a preferred method of the present invention and/or embodiments thereof the level of expression of at least one biomarker selected from the group consisting of CMPK1, PRKACA, EML4, GANAB, PRKAR1A, PSME2, STX5, MDH1, OTUB1, MGP, is down-regulated in said sample and is indicative of the treatment not being effective.

In a preferred method of the present invention and/or embodiments thereof the level of expression of at least one biomarker selected from the group consisting of CMPK1, APIP, STX5, AASDHPPT, MARCKSL1, PRKACA, PRKACB, EML4, GANAB, RAB1A is down-regulated in said sample and is indicative of the treatment not being effective.

In a preferred method of the present invention and/or embodiments thereof the level of expression of at least one biomarker selected from the group consisting of CMPK1, PRKACA, EML4, GANAB, PRKAR1A is down-regulated in said sample and is indicative of the treatment not being effective.

In a preferred method of the present invention and/or embodiments thereof the treatment is selected from the group consisting of chemotherapy, biological therapy, and/or radiotherapy and/or combinations thereof. For example a novel chemotherapy is test, or a antibody, or a combination thereof. Also combination of known therapies is envisioned, such as a combination of known chemotherapeutics, or in combination with X-ray radiation therapy and/or targeted antibodies.

The present invention is also directed to a method to screen for compounds for treatment of triple negative breast cancer using at least one biomarker selected from the group consisting of CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDCl₂₃, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1.

In a preferred method of the present invention and/or embodiments thereof an assay is used that determines the expression level of the biomarker.

Preferably a compound is selected that upregulates the expression level of at least one biomarker selected from the group consisting of CMPK1, PRKACA; PRKACB, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM and/or a compound that down-regulates the expression level of at least one biomarker selected from the group consisting of PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, UBE2Q1, SMC4, and/or HAPLN1.

Preferably a compound is selected that down-regulates the expression level of at least one biomarker selected from the group consisting of GPRC5A, LPCAT1, ACTBL2, SIGMAR1, CPT1A, SFXN2, RBBP7, BAZ1B, PPOX, FLAD1, MIF, FDPS, C8orf55, KTN1, GTPBP4.

Preferably a compound is selected that down-regulates the expression level of at least one biomarker selected from the group consisting of MIF, FDPS, ACTBL2, KTN1, C8orf55, GTPBP4, RBBP7, FLAD1, PPOX.

Preferably a compound is selected that down-regulates the expression level of at least one biomarker selected from the group consisting of GPRC5A, LPCAT1, ACTBL2, SIGMAR1, CPT1A, PPOX, FLAD1, MIF, FDPS.

Preferably a compound is selected that down-regulates the expression level of at least one biomarker selected from the group consisting of GPRC5A, LPCAT1, ACTBL2, PPOX, FLAD1.

Preferably a compound is selected that down-regulates the expression level of at least one biomarker selected from the group consisting of ACTBL2, PPOX, FLAD1.

Preferably a compound is selected that upregulates the expression level of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A.

Preferably a compound is selected that upregulates the expression level of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A. Preferably a compound is selected that upregulates the expression level of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A.

Preferably a compound is selected that upregulates the expression level of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A.

Preferably a compound is selected that upregulates the expression level of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A.

Preferably a compound is selected that upregulates the expression level of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A.

Preferably a compound is selected that upregulates the expression level of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A.

Preferably a compound is selected that upregulates the expression level of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A is. Preferably the biomarker is not FTH1, and/or TF and/or YWHAQ.

Preferably a compound is selected that upregulates the expression level of at least one biomarker selected from the group consisting of CMPK1, PRKACA, EML4, GANAB, PRKAR1A, PSME2, STX5, MDH1, FTH1, OTUB1, MGP, TF.

Preferably a compound is selected that upregulates the expression level of at least one biomarker selected from the group consisting of CMPK1, PRKACA, EML4, GANAB, PRKAR1A, PSME2, STX5, MDH1, OTUB1, MGP, TF.

Preferably a compound is selected that upregulates the expression level of at least one biomarker selected from the group consisting of CMPK1, PRKACA, EML4, GANAB, PRKAR1A, PSME2, STX5, MDH1, FTH1, OTUB1, MGP.

Preferably a compound is selected that upregulates the expression level of at least one biomarker selected from the group consisting of CMPK1, PRKACA, EML4, GANAB, PRKAR1A, PSME2, STX5, MDH1, OTUB1, MGP.

Preferably a compound is selected that upregulates the expression level of at least one biomarker selected from the group consisting of CMPK1, APIP, STX5, AASDHPPT, MARCKSL1, PRKACA, PRKACB, EML4, GANAB, RAB1A.

Preferably a compound is selected that upregulates the expression level of at least one biomarker selected from the group consisting of CMPK1, PRKACA, EML4, GANAB, PRKAR1A.

In a preferred method of the present invention and/or embodiments thereof compounds are screened that bind to at least one of the biomarker selected from the group consisting of CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDCl₂₃, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1.

The present invention is additionally directed to a kit for determining a prognosis, a treatment, and/or the effectiveness of a treatment for a patient with triple negative breast cancer, wherein said kit comprises a compound capable of detecting the level of expression of at least one biomarker selected from the group of CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1 in a biological sample.

In a preferred method, use, or kit according to the invention and/or embodiments thereof the biomarker is selected from the group of CMPK1, PRKACA, PRKACB, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM. Preferably the biomarker is selected from the group of CMPK1, PRKACA, PRKACB, EML4, GANAB, PSME2, PRKAR1A, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM. Preferably the biomarker is selected from the group of the biomarker is selected from the group consisting of CMPK1, PRKACA, PRKACB, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM. Preferably the biomarker is selected from the group consisting of the biomarker is selected from the group of CMPK1, PRKACA, PRKACB, EML4, GANAB, PSME2, PRKAR1A, MDH1, OTUB1, TF, DPYSL2, MGP, CAPZB, ATP5D, SP100, NDRG2, CYB5B, STIP1, TNKS1BP1, SPATS2L, PRKCSH, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDCl₂₃, NUDC, GYG1, PGD, AASDHPPT, STX5, CSTB, MARCKSL1, LRP1, PSME1, APIP, GBP1, BLM.

In a preferred method, use, or kit according to the invention and/or embodiments thereof the biomarker is not FTH1 and/or not YWHAQ.

In a preferred method, use, or kit according to the invention and/or embodiments thereof the biomarker is the biomarker is selected from the group of CMPK1, PRKACA, PRKAR1A, CYB5B, AP1G1, AIFM1, TF, FTH1, MIF, PRKCSH, FDPS, CFL1, PSMA1, YWHAQ, STIP1, PSMC2, MDH1, CAPZB, RAB1A, GANAB, DPYSL2, ACTBL2, KTN1, C8orf55, OTUB1, TUBA1C, HNRNPUL1, GTPBP4, TNKS1BP1, EML4, ATP5D, RBBP7, GLG1, AHCYL1, CSNK2A1, EWSR1, PSME2, MARCKSL1, KIAA0174, FLAD1, HLA-C, UBE2Q1, PSMB9, SP100, SPATS2L, AGL, GOSR1, NDRG2, PTK2, MGP, SMC4, PPOX, HAPLN1, STX5, SKIV2L, GSTM1. Preferably the biomarker is selected from the group consisting of CMPK1, PRKACA, PRKAR1A, CYB5B, AP1G1, AIFM1, TF, MIF, PRKCSH, FDPS, CFL1, PSMA1, YWHAQ, STIP1, PSMC2, MDH1, CAPZB, RAB1A, GANAB, DPYSL2, ACTBL2, KTN1, C8orf55, OTUB1, TUBA1C, HNRNPUL1, GTPBP4, TNKS1BP1, EML4, ATP5D, RBBP7, GLG1, AHCYL1, CSNK2A1, EWSR1, PSME2, MARCKSL1, KIAA0174, FLAD1, HLA-C, UBE2Q1, PSMB9, SP100, SPATS2L, AGL, GOSR1, NDRG2, PTK2, MGP, SMC4, PPOX, HAPLN1, STX5, SKIV2L, GSTM1. Preferably the biomarker is selected form the group consisting of CMPK1, PRKACA, PRKAR1A, CYB5B, AP1G1, AIFM1, TF, FTH1, MIF, PRKCSH, FDPS, CFL1, PSMA1, STIP1, PSMC2, MDH1, CAPZB, RAB1A, GANAB, DPYSL2, ACTBL2, KTN1, C8orf55, OTUB1, TUBA1C, HNRNPUL1, GTPBP4, TNKS1BP1, EML4, ATP5D, RBBP7, GLG1, AHCYL1, CSNK2A1, EWSR1, PSME2, MARCKSL1, KIAA0174, FLAD1, HLA-C, UBE2Q1, PSMB9, SP100, SPATS2L, AGL, GOSR1, NDRG2, PTK2, MGP, SMC4, PPOX, HAPLN1, STX5, SKIV2L, GSTM1. Preferably the biomarker is selected from the group consisting of CMPK1, PRKACA, PRKAR1A, CYB5B, AP1G1, AIFM1, TF, MIF, PRKCSH, FDPS, CFL1, PSMA1, STIP1, PSMC2, MDH1, CAPZB, RAB1A, GANAB, DPYSL2, ACTBL2, KTN1, C8orf55, OTUB1, TUBA1C, HNRNPUL1, GTPBP4, TNKS1BP1, EML4, ATP5D, RBBP7, GLG1, AHCYL1, CSNK2A1, EWSR1, PSME2, MARCKSL1, KIAA0174, FLAD1, HLA-C, UBE2Q1, PSMB9, SP100, SPATS2L, AGL, GOSR1, NDRG2, PTK2, MGP, SMC4, PPOX, HAPLN1, STX5, SKIV2L, GSTM1.

In a preferred method, use, or kit according to the invention and/or embodiments thereof the biomarker is selected from the group of CMPK1, PRKACA, PRKAR1A, CYB5B, TF, FTH1, MIF, PRKCSH, FDPS, YWHAQ, STIP1, MDH1, CAPZB, GANAB, DPYSL2, ACTBL2, KTN1, C8orf55, OTUB1, GTPBP4, TNKS1BP1, EML4, ATP5D, RBBP7, GLG1, PSME2, MARCKSL1, FLAD1, SP100, SPATS2L, NDRG2, MGP, PPOX, STX5. Preferably the biomarker is selected from the group consisting of CMPK1, PRKACA, PRKAR1A, CYB5B, TF, MIF, PRKCSH, FDPS, YWHAQ, STIP1, MDH1, CAPZB, GANAB, DPYSL2, ACTBL2, KTN1, C8orf55, OTUB1, GTPBP4, TNKS1BP1, EML4, ATP5D, RBBP7, GLG1, PSME2, MARCKSL1, FLAD1, SP100, SPATS2L, NDRG2, MGP, PPOX, STX5. Preferably the biomarker is selected from the group consisting of CMPK1, PRKACA, PRKAR1A, CYB5B, TF, FTH1, MIF, PRKCSH, FDPS, STIP1, MDH1, CAPZB, GANAB, DPYSL2, ACTBL2, KTN1, C8orf55, OTUB1, GTPBP4, TNKS1BP1, EML4, ATP5D, RBBP7, GLG1, PSME2, MARCKSL1, FLAD1, SP100, SPATS2L, NDRG2, MGP, PPOX, STX5. Preferably the biomarker is selected from the group consisting of CMPK1, PRKACA, PRKAR1A, CYB5B, TF, MIF, PRKCSH, FDPS, STIP1, MDH1, CAPZB, GANAB, DPYSL2, ACTBL2, KTN1, C8orf55, OTUB1, GTPBP4, TNKS1BP1, EML4, ATP5D, RBBP7, GLG1, PSME2, MARCKSL1, FLAD1, SP100, SPATS2L, NDRG2, MGP, PPOX, STX5.

In a preferred method, use, or kit according to the invention and/or embodiments thereof the biomarker is selected from the group of CMPK1, PRKACA, PRKAR1A, CYB5B, AP1G1, AIFM1, TF, FTH1, MIF, PRKCSH, FDPS, CFL1, PSMA1, YWHAQ, STIP1, PSMC2, MDH1, CAPZB, RAB1A, GANAB, DPYSL2, ACTBL2, KTN1, C8orf55, OTUB1, TUBA1C, HNRNPUL1, GTPBP4, TNKS1BP1, EML4, ATP5D, RBBP7, GLG1, AHCYL1, CSNK2A1, EWSR1, PSME2, MARCKSL1, KIAA0174, FLAD1. In a preferred method, use, or kit according to the invention and/or embodiments thereof the biomarker is selected from the group of CMPK1, PRKACA, PRKAR1A, CYB5B, AP1G1, AIFM1, TF, MIF, PRKCSH, FDPS, CFL1, PSMA1, YWHAQ, STIP1, PSMC2, MDH1, CAPZB, RAB1A, GANAB, DPYSL2, ACTBL2, KTN1, C8orf55, OTUB1, TUBA1C, HNRNPUL1, GTPBP4, TNKS1BP1, EML4, ATP5D, RBBP7, GLG1, AHCYL1, CSNK2A1, EWSR1, PSME2, MARCKSL1, KIAA0174, FLAD1. In a preferred method, use, or kit according to the invention and/or embodiments thereof the biomarker is selected from the group of CMPK1, PRKACA, PRKAR1A, CYB5B, AP1G1, AIFM1, TF, FTH1, MIF, PRKCSH, FDPS, CFL1, PSMA1, STIP1, PSMC2, MDH1, CAPZB, RAB1A, GANAB, DPYSL2, ACTBL2, KTN1, C8orf55, OTUB1, TUBA1C, HNRNPUL1, GTPBP4, TNKS1BP1, EML4, ATP5D, RBBP7, GLG1, AHCYL1, CSNK2A1, EWSR1, PSME2, MARCKSL1, KIAA0174, FLAD1. In a preferred method, use, or kit according to the invention and/or embodiments thereof the biomarker is selected from the group of CMPK1, PRKACA, PRKAR1A, CYB5B, AP1G1, AIFM1, TF, MIF, PRKCSH, FDPS, CFL1, PSMA1, STIP1, PSMC2, MDH1, CAPZB, RAB1A, GANAB, DPYSL2, ACTBL2, KTN1, C8orf55, OTUB1, TUBA1C, HNRNPUL1, GTPBP4, TNKS1BP1, EML4, ATP5D, RBBP7, GLG1, AHCYL1, CSNK2A1, EWSR1, PSME2, MARCKSL1, KIAA0174, FLAD1.

In a preferred method, use, or kit according to the invention and/or embodiments thereof the biomarker is selected from the group of CMPK1, PRKACA; PRKACB, EML4, GANAB, PPOX, PSME2, PRKAR1A, FTH1, MDH1, OTUB1, FLAD1, TF, DPYSL2, APIP, GPRC5A, LPCAT1, ACTBL2, STX5, AASDHPPT, SIGMAR1. In a preferred method, use, or kit according to the invention anchor embodiments thereof the biomarker is selected from the group of CMPK1, PRKACA; PRKACB, EML4, GANAB, PPOX, PSME2, PRKAR1A, MDH1, OTUB1, FLAD1, TF, DPYSL2, APIP, GPRC5A, LPCAT1, ACTBL2, STX5, AASDHPPT, SIGMAR1.

In a preferred method, use, or kit according to the invention and/or embodiments thereof the biomarker is selected from the group of ACTBL2, BLM, CPT1A, GBP1, GPRC5A, LPCAT1, AK3, APIP, BDH1, PSME1, LRP1, MARCKSL1, MGP, ACTL8, NDRG2, SPATS2L, DPYSL2, PPOX, FTH1, PSME2, FLAD1.

In a preferred method, use, or kit according to the invention and/or embodiments thereof the biomarker is selected from the group of CMPK1, PRKACA, EML4, GANAB, PPOX, PRKAR1A, PSME2, STX5, MDH1, FTH1, OTUB1, MGP, TF, ACTBL2, FLAD1. (top 15 protein).

In a preferred method, use, or kit according to the invention and/or embodiments thereof the biomarker is selected from the group of CMPK1, PRKACA, EML4, GANAB, PPOX, PRKAR1A, PSME2, STX5, MDH1, OTUB1, MGP, TF, ACTBL2, FLAD1.

In a preferred method, use, or kit according to the invention and/or embodiments thereof the level of expression of at least one biomarker selected from the group consisting of GPRC5A, LPCAT1, ACTBL2, SIGMAR1, CPT1A, SFXN2, RBBP7, BAZ1B, PPOX, FLAD1, MIF, FDPS, C8orf55, KTN1, GTPBP4.

In a preferred method, use, or kit according to the invention and/or embodiments thereof the level of expression of at least one biomarker selected from the group consisting of MIF, FDPS, ACTBL2, KTN1, C8orf55, GTPBP4, RBBP7, FLAD1, PPOX.

In a preferred method, use, or kit according to the invention and/or embodiments thereof the level of expression of at least one biomarker selected from the group consisting of GPRC5A, LPCAT1, ACTBL2, SIGMAR1, CPT1A, PPOX, FLAD1, MIF, FDPS.

In a preferred method, use, or kit according to the invention and/or embodiments thereof the level of expression of at least one biomarker selected from the group consisting of GPRC5A, LPCAT1, ACTBL2, PPOX, FLAD1.

In a preferred method, use, or kit according to the invention and/or embodiments thereof the level of expression of at least one biomarker selected from the group consisting of ACTBL2, PPOX, FLAD1.

In a preferred method, use, or kit according to the invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A.

In a preferred method, use, or kit according to the invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, YWHAQ, RAB1A.

In a preferred method, use, or kit according to the invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A.

In a preferred method, use, or kit according to the invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A.

In a preferred method, use, or kit according to the invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, YWHAQ, RAB1A.

In a preferred method, use, or kit according to the invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, TF, HNRNPUL1, PSMC2, DPYSL2, CAPZB, CYB5B, CFL1, STIP1, TNKS1BP1, PSMA1, PRKCSH, RAB1A.

In a preferred method, use, or kit according to the invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, FTH1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A.

In a preferred method, use, or kit according to the invention and/or embodiments thereof the level of expression of at least one biomarker selected from the group consisting of CMPK1, APIP, STX5, AASDHPPT, MARCKSL1, BLM, LRP1, GYG1, GBP1, NUDC, PRKACA, PRKACB, EML4, GANAB, PSME2, PRKAR1A, AIFM1, MDH1, OTUB1, AP1G1, TUBA1C, HNRNPUL1, PSMC2, DPYSL2, CAPZB, CYB5B, CFL1, STIP1, TNKS1BP1, PSMA1, PRKCSH, RAB1A is. Preferably the biomarker is not FTH1, and/or TF and/or YWHAQ.

In a preferred method, use, or kit according to the invention and/or embodiments thereof the level of expression of at least one biomarker selected from the group consisting of CMPK1, PRKACA, EML4, GANAB, PRKAR1A, PSME2, STX5, MDH1, FTH1, OTUB1, MGP, TF.

In a preferred method, use, or kit according to the invention and/or embodiments thereof the level of expression of at least one biomarker selected from the group consisting of CMPK1, PRKACA, EML4, GANAB, PRKAR1A, PSME2, STX5, MDH1, OTUB1, MGP, TF.

In a preferred method, use, or kit according to the invention and/or embodiments thereof the level of expression of at least one biomarker selected from the group consisting of CMPK1, PRKACA, EML4, GANAB, PRKAR1A, PSME2, STX5, MDH1, FTH1, OTUB1, MGP.

In a preferred method, use, or kit according to the invention and/or embodiments thereof the level of expression of at least one biomarker selected from the group consisting of CMPK1, PRKACA, EML4, GANAB, PRKAR1A, PSME2, STX5, MDH1, OTUB1, MGP.

In a preferred method, use, or kit according to the invention and/or embodiments thereof the level of expression of at least one biomarker selected from the group consisting of CMPK1, APIP, STX5, AASDHPPT, MARCKSL1, PRKACA, PRKACB, EML4, GANAB, RAB1A.

In a preferred method, use, or kit according to the invention and/or embodiments thereof the level of expression of at least one biomarker selected from the group consisting of CMPK1, PRKACA, EML4, GANAB, PRKAR1A.

In a preferred method, use, or kit of the present invention and/or embodiments thereof the biomarker is selected from the group consisting of FTH1, CMPK1, AIFM1, MTHFD1, EML4, GANAB, AP1G1, CTNNA1, STX12, CAPZB, and/or AP1M1. In a preferred method, use, or kit of the present invention and/or embodiments thereof the biomarker is selected from the group consisting of MTHFD1, AP1G1, CTNNA1, STX12, CAPZB, and/or AP1M1.

In a preferred method, use, or kit of the present invention and/or embodiments thereof the biomarker is selected from the group consisting of MTHFD1, CTNNA1, STX12, and/or AP1M1.

In a preferred method, use, or kit of the present invention and/or embodiments thereof the biomarker is selected from the group consisting of AP1G1, and/or CAPZB.

In a preferred method, use, or kit of the present invention and/or embodiments thereof the biomarker is AP1G1 and at least one biomarker selected from the group consisting of FTH1, CMPK1, AIFM1, MTHFD1, EML4, GANAB, CTNNA1, STX12, CAPZB, and/or AP1M1. In a preferred method, use, or kit of the present invention and/or embodiments thereof the biomarker is AP1G1, and at least one biomarker selected from the group consisting of MTHFD1, CTNNA1, STX12, CAPZB, and/or AP1M1.

In a preferred method, use, or kit of the present invention and/or embodiments thereof the biomarker is AP1G1 and at least one biomarker selected from the group consisting of MTHFD1, CTNNA1, STX12, and/or AP1M1.

In a preferred method, use, or kit of the present invention and/or embodiments thereof the biomarker is AP1G1 and CAPZB.

In a preferred method, use, or kit of the present invention and/or embodiments thereof the biomarker is AP1G1 and at least one biomarker selected from the group consisting of CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1.

In a preferred method, use, or kit of the present invention and/or embodiments thereof the biomarker is CAPZB and at least one selected from the group consisting of FTH1, CMPK1, AIFM1, MTHFD1, EML4, GANAB, AP1G1, CTNNA1, STX12, and/or AP1M1. In a preferred method, use, or kit of the present invention and/or embodiments thereof the biomarker is CAPZB and at least one biomarker selected from the group consisting of MTHFD1, AP1G1, CTNNA1, STX12, and/or AP1M1.

In a preferred method, use, or kit of the present invention and/or embodiments thereof the biomarker is CAPZB and at least one biomarker selected from the group consisting of MTHFD1, CTNNA1, STX12, and/or AP1M1.

In a preferred method, use, or kit of the present invention and/or embodiments thereof the biomarker is CAPZB and at least one biomarker selected from the group consisting of CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1

In a preferred method, use, or kit of the present invention and/or embodiments thereof the biomarker is AP1G1 and CAPZB and at least one biomarker selected from the group consisting of FTH1, CMPK1, AIFM1, MTHFD1, EML4, GANAB, CTNNA1, STX12, CAPZB, and/or AP1M1. In a preferred method, use, or kit of the present invention and/or embodiments thereof the biomarker is AP1G1 and CAPZB and at least one biomarker selected from the group consisting of MTHFD1, CTNNA1, STX12, and/or AP1M1.

In a preferred method, use, or kit of the present invention and/or embodiments thereof the biomarker is AP1G1 and CAPZB and at least one biomarker selected from the group consisting of MTHFD1, CTNNA1, STX12, and/or AP1M1.

In a preferred method, use, or kit of the present invention and/or embodiments thereof the biomarker is AP1G1 and CAPZB and at least one biomarker selected from the group consisting of CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1.

In a preferred method, use, or kit of the present invention and/or embodiments thereof the biomarker is CMPK1, AIFM1, FTH1, EML4, GANAG, AP1G1, and CAPZB. In a preferred method, use, or kit of the present invention and/or embodiments thereof the biomarker is EML4, AP1G1, STX12, and CAPZB. In a preferred method, use, or kit of the present invention and/or embodiments thereof the biomarker is EML4, AP1G1, and CAPZB. In a preferred method, use, or kit of the present invention and/or embodiments thereof the biomarker is CMPK1, AIFM1, FTH1, AP1G1, AP1M1, and CAPZB. In a preferred method, use, or kit of the present invention and/or embodiments thereof the biomarker is CMPK1, AIFM1, FTH1, AP1G1, and CAPZB. In a preferred method, use, or kit of the present invention and/or embodiments thereof the biomarker is AP1G1 and CAPZB.

In a preferred method, use, or kit according to the invention and/or embodiments thereof the biomarker is CMPK1, FTH1, and/or YWHAQ. Preferably the biomarker is CMPK1.

In a preferred method, use, or kit according to the invention and/or embodiments thereof CMPK1 is up regulated.

In a preferred method, use, or kit according to the invention and/or embodiments thereof at least 2, preferably at least 3, more preferably at least 4, 5, 7, 10, 12, 15, 17, or 20 biomarkers are used.

In a preferred method, use, or kit according to the invention and/or embodiments thereof A biomarker may be a protein, nucleic acid encoding for a protein, peptides of a protein, fragments of protein, or mutants thereof, and or metabolites. Fragments or mutants preferably have at least 70% sequence identity to the biomarker as disclosed herein. More preferably at least 75% sequence identity, more preferably at least 80% sequence identity, more preferably at least 85% sequence identity, more preferably at least 90% sequence identity, more preferably at least 92% sequence identity, more preferably at least 94% sequence identity, more preferably at least 95% sequence identity, more preferably at least 97% sequence identity, more preferably at least 99% sequence identity. Preferred biomarkers are proteins, peptides, or nucleic acids coding for a peptide or protein, or fragments and/or mutants thereof. Most preferred biomarkers are peptides and/or proteins and/or mutants and/or fragments of these peptides and/or proteins.

In a preferred method, use, or kit according to the invention and/or embodiments thereof the method uses a technique selected from the group consisting of mass spectrometry, DNA array, immunohistochemistry, antibodies, and-or probes. Preferably the technique is a multiplex technique.

In a preferred method, use, or kit according to the invention and/or embodiments thereof the biological sample is selected from tumor cells, tissue, blood, serum, urine, nipple aspirate fluid, circulating tumor cells, cerebrospinal fluid, aerosol, and/or thrombocytes.

In a preferred method, use, or kit according to the invention and/or embodiments thereof the prognosis is development of metastasis.

Experimental

Patients and Tumor Tissues

63 fresh frozen primary breast cancer (BC) tissues from our liquid N2 bank were selected. Primary tumors were removed from patients who did not receive any adjuvant and advanced hormonal therapy and chemotherapy, and were diagnosed with local and distance relapse at same time points. Those patients were diagnosed as triple negative breast cancer (TNBC) phenotype based on negative message RNA expression of estrogen (ER, <0.2), progesterone (PgR, <0.1) and human epidermal growth factor receptor 2 (HER2, <18.0) using quantitative polymerase chain reaction (qPCR). Tumor tissues were further divided into two classes based on clinical metastatic status of corresponding patients during the period of clinical follow-up:

(1) patients who developed local and distant relapse within 60 months were defined as having poor prognosis;

(2) patients exempted from clinical metastasis for at least 60 month were classified into favorable prognostic group.

For quality control of LC-MS/MS profiling, we used microscopically inspected BC tumors containing multiple cell types as a control sample.

This study was approved by the Medical Ethics Committee of the Erasmus Medical Center Rotterdam, The Netherlands (MEC 02.953) and was performed in accordance to the Code of Conduct of the Federation of Medical Scientific Societies in The Netherlands, and wherever possible we adhered to the Reporting Recommendations for Tumor Marker Prognostic Studies (REMARK).

1.2 Clinical Histopathological Features of TNBC Cases

Histopathological characterization of 63 TNBC tumor samples was determined by a pathologist mainly based on haemotoxylin-eosin (HE) stained formalin-fixed paraffin-embedded sections and partially based on HE-stained cryosections of corresponding tumor material. Majority of tumors used in this study were classified as invasive ductal carcinoma (IDC) and high pathological grade (grade 3).

1.3 Isolation of TNBC Cells by LCM and Sample Preparation

Isolation of tumor cells was performed using an in-house optimized protocol of cryosectioning followed by laser capture microdissection (LCM) based on previously documented procedure (Umar, A., et al. Identification of a putative protein profile associated with tamoxifen therapy resistance in breast cancer. Mol Cell Proteomics 8, 1278-1294 (2009)). Cryosectioning was performed as described below: 8 μm tissue cryosections were fixed in ice-cold 70% ethanol, dehydrated in 100% ethanol and stored in −80° C. until haematoxylin staining using in house protocol. The slides were briefly washed in tap water, stained for 30 s in haematoxylin, washed again in tap water, subsequently dehydrated in 50%, 70%, 95% and twice 100% ethanol for 15 s each and 60 s for the final 100% ethanol step, and were subsequently air-dried. A volume of 100 μl Halt protease and phosphatase inhibitor cocktail (Thermo scientific, Rockford, Ill., USA) was added into tap water, 50% and 70% ethanol respectively to inhibit non-specific cleavage caused by endogenous enzymes within the duration of LCM. The LCM was performed right after staining using a P.A.L.M. LCM device (type P-MB, P.A.L.M. Microlaser Technologies AG, Bernried, Germany). For each cryosection an area of 500,000 μm² equivalent to ˜4,000 tumor epithelial cells (Number of cells=dissected area×thickness of cyosection/1,000 μm³ cell volume) was collected in ZEISS opaque adhesive caps (Carl Zeiss MicroImaging GmbH, Munich, Germany). Dissected debris was gently suspended in 20 μl of 0.1% RapiGest (Waters Corp., Milford, Mass.) and then kept in 0.5-ml Eppendorf LoBind tubes (Eppendorf, Hamburg, Germany). Collected cells were stored at −80° C. until further processing. Two types of control samples were processed together with TNBC samples: (1) 5 biological replicate controls, named as LCM controls, were microdissected with above-mentioned protocol through the duration of TNBC tissue microdissection; (2) 12 technical replicate controls, named as whole tissue lysate (WTL) controls, were prepared from tissue lysates of the same tissue as LCM controls. Due to trace amount of microdissected cells used in this investigation, protein concentration was under the detection limits of any available protein assay, we therefore roughly estimated protein concentration based on dissected tissue area (i.e. ˜4,000 cells corresponds to ˜400 ng of total protein). The protein concentration of WTL control samples were extrapolated through bicinchoninic acid (BCA) protein assay and diluted into a final concentration of 100 ng/μl.

Microdissected TNBC, LCM control and WTL control samples were fully randomized and divided into two batches for digestion processing. Protein digestion was performed following in house optimized in-solution protein digestion protocol as described below. Briefly, cells were lysed by sonication in RapiGest solution using an Ultrasonics Disruptor Sonifier II (Model W-250/W-450, Branson Ultrasonics, Danbury, Conn.) for 1 min at 70% amplitude. Proteins were subsequently denatured at 95° C. for 5 min. Denatured proteins were further reduced at 60° C. for 30 min using 1 μl of 5 mM dithiothreitol (DTT) (SIGMA, Saint Louis, Mo., USA), and alkylated in the dark for 30 min with iodoacetamide (IAA) (Thermo scientific, Rockford, Ill., USA). Fully unfolded proteins were processed for 4 h tryptic digestion at 37° C. in accordance with the instructions of the manufacturer using MS-grade porcine modified trypsin gold (Promega, Madison, Wis., USA) at a 1:20 (w/v) ratio as described previously. Digestion was terminated by incubation together with 0.5% Trifluoroacetic acid (TFA) at 37° C. for 30 min. Undissolved cellular debris was removed by centrifugation at 14,000 rpm for 15 min, and supernatant were transferred to a new Eppendorf Lobind tube and stored at −80° C. until MS measurement.

Prior to nLC-MS/MS analysis, peptide mixture solution was thawed at room temperature and precipitates formed during storage were spun down again at 14,000 rpm for 15 min. Of each peptide sample 23 μl was transferred to HPLC vials.

1.4 Nano Liquid Chromatography and High Resolution Tandem Mass Spectrometry

Nano-LC-Orbitrap-MS/MS was performed on a nLC system (Ultimate 3000, Dionex, Amsterdam, The Netherlands) hyphenated online with a hybrid linear ion trap/Orbitrap mass spectrometer ((LTQ-Orbitrap-XL, ThermoElectron, Bremen, Germany) following a slightly modified procedure as described previously [8]. For each sample, a volume of 20 μl (equivalent to ˜4,000 cells or 400 ng) was firstly loaded on a trap column (PepMap C18, 300 μm I.D.×5 mm, 5 μm particle size, 100 Å pore size; Dionex, Amsterdam, The Netherlands) for concentration and desalting using 0.1% TFA (in water) as loading solvent at a flow rate of 20 μl/min. The trap column was then switched online to directly connect with a reversed-phase (RP) 75-μm I.D.×50-cm fused silica capillary column packed with 3 μm C18 particles (PepMap, Dionex, Amsterdam, The Netherlands) and peptides were gradually eluted out with a flow rate of 250 nl/min at 40° C. column temperature using the following binary gradient: The gradient started with 100% mobile phase A (97.9% H₂O, 2% acetonitrile, 0.1% formic acid) to 25% mobile phase B (80% acetonitrile, 19.02% H₂O, 0.08% formic acid) over the first 120 min, and then a steeper gradient was used to further increase mobile phase B to 50% in the next 60 min. The eluted peptides were directly sprayed with a voltage of 1.6 kV into the on-line coupled LTQ-Orbitrap-XL MS using electro-spray ionization (ESI) equipped with a metal-coated nano ESI emitters (New Objective, Woburn, Mass.). Mass spectra were acquired over the range mass-to-charge ratio (m/z) range 400-1,800 at a resolving power of 30,000 at 400 m/z. Target of automatic gain (AGC) were set at 10⁶ ions and mass was locked at 445.120025 u protonated with (Si(CH₃)₂O))₆). On the basis of this, full scan top 5 intensive ions were consecutively isolated (AGC target set to 10⁴ ions) and fragmented by collisional activated dissociation (CAD) applying 35% normalized collision energy in the linear ion trap. Parent ions within a mass window of ±5 ppm or dissociation were then excluding for MS/MS fragmentation in next 3 min or until the precursor intensity fell below a signal-to-noise ratio (S/N) of 1.5 for more than 10 scans (early expiration). Full scan and MS/MS fragmentation spectra were partially simultaneously acquired in Orbtitrap and linear ion trap parts.

1.5 Identification, Quantitation and Filtering of Peptides

The recorded MS spectra were analyzed by MaxQuant Software (Cox, J. & Mann, M. MaxQuant enables high peptide identification rates, individualized p.p.b.-range mass accuracies and proteome-wide protein quantification. Nat Biotechnol 26, 1367-1372 (2008)) (version 1.1.1.36). To construct the MS/MS peak list file, up to top 8 peaks per 100 Da window were extracted and submitted to search against a concatenated forward and reverse version of the UniProtKB/Swiss-Prot human database (generated from version 2011_{—}03), as well as a database constructed with common present contaminants. An initial precursor mass window was set at 20 ppm with a fragment mass window of 0.5 Th for database searching. Carbamidomethylation of cysteines was defined as fixed modification, while protein N-terminal acetylation and methionine oxidation were defined as variable modifications for the database searching. The cutoff of global false discovery rate (FDR) for peptide identification was set to 0.01, and only the peptides with ≧7 amino acid residues were included for identification.

Label-free quantitation was performed in MaxQuant for the identified peptides [Luber, C. A., et al. Quantitative proteomics reveals subset-specific viral recognition in dendritic cells. Immunity 32, 279-289 (2010)]. A retention time window of 10 min was applied to match the same accurate masses between multiple LC-MS/MS runs. An option of second identifications was selected to allow identifying the co-eluted peptides from given MS/MS spectra [Cox, J., et al. Andromeda—a peptide search engine integrated into the MaxQuant environment. Journal of proteome research 10, 1794-1805 (2011)].

Additional filtering steps were performed on the peptides posterior to identification. The local FDR index, posterior error probability (PEP) score, was stringently restricted <0.05 to preserve the confidently identified peptides. Peptides identified with reversed sequences from sequence library and peptides assigned to contaminants were also removed from further analysis. Furthermore, only unique peptides were reserved. Finally, to improve accuracy of protein quantification and statistical power, only peptides with at least 20 observations out of 63 samples were included for further analysis.

1.6 Data Analysis and Statistics

Raw peptide abundance of 63 TNBC samples calculated from label-free quantitation as described above was analyzed by the R language based statistical tool DanteR (v1.0.1.1) [Polpitiya, A. D., et al. DAnTE: a statistical tool for quantitative analysis of -omics data. Bioinformatics (Oxford, England) 24, 1556-1558 (2008)]. The raw abundance was first converted by log₂ transformation and then normalized based on the median center of the abundance distribution to remove bias introduced by technical reasons (e.g. slight variation of numbers of tumor cells, incorrect pipette volumes and injection error). To find differentially expressed proteins, a mixed-effect analysis of variance model (ME-ANOVA) was selected to analyze significance as well as log₂ fold changes of identified proteins between favorable and adverse prognostic tumors by using the formula: y=experimental+group+peptide+error. Up to 10 most abundant peptides assigned to a certain protein were taken into account in ME-ANOVA test. ME-ANOVA reference may be found in
Daly, D. S., et al. Mixed-effects statistical model for comparative LC-MS proteomics studies. Journal of proteome research 7, 1209-1217 (2008).
Karpievitch, Y. V., et al. Normalization of peak intensities in bottom-up MS-based proteomics using singular value decomposition. Bioinformatics (Oxford, England) 25, 2573-2580 (2009).
Oberg, A. L. & Vitek, O, Statistical design of quantitative mass spectrometry-based proteomic experiments. Journal of proteome research 8, 2144-2156 (2009).
Bukhman, Y. V., et al. Design and analysis of quantitative differential proteomics investigations using LC-MS technology. Journal of bioinformatics and computational biology 6, 107-123 (2008).
Clough, T., et al. Protein quantification in label-free LC-MS experiments. Journal of proteome research 8, 5275-5284 (2009).
Oberg, A. L., et al. Statistical analysis of relative labeled mass spectrometry data from complex samples using ANOVA. Journal of proteome research 7, 225-233 (2008).

Calculated p-values of identified proteins were further corrected by Benjamini-Hochberg correction to remove false positive hits [Benjamini, Y. & Hochberg, Y. CONTROLLING THE FALSE DISCOVERY RATE—A PRACTICAL AND POWERFUL APPROACH TO MULTIPLE TESTING. J. R. Stat. Soc. Ser. B-Methodol. 57, 289-300 (1995)]. Differentially abundant proteins with a threshold of p<0.05 were then pooled out in the form of abundance of relevant peptides. To estimate the abundance of differentially expressed proteins, Z-score normalization was performed on un-imputed peptides assigned to the given proteins across the samples using the formula: (value−mean)/standard deviation.

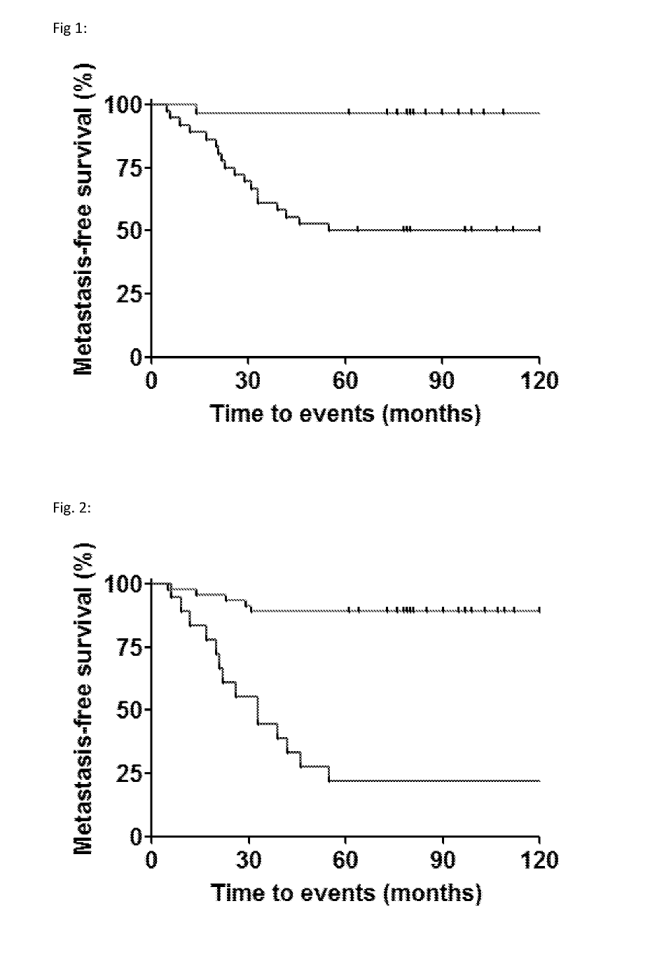

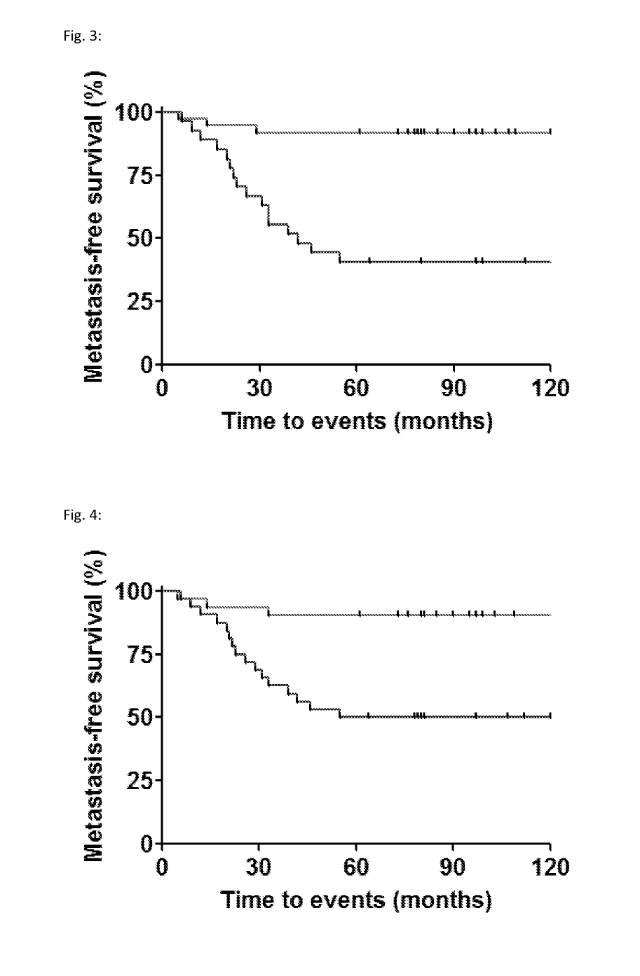

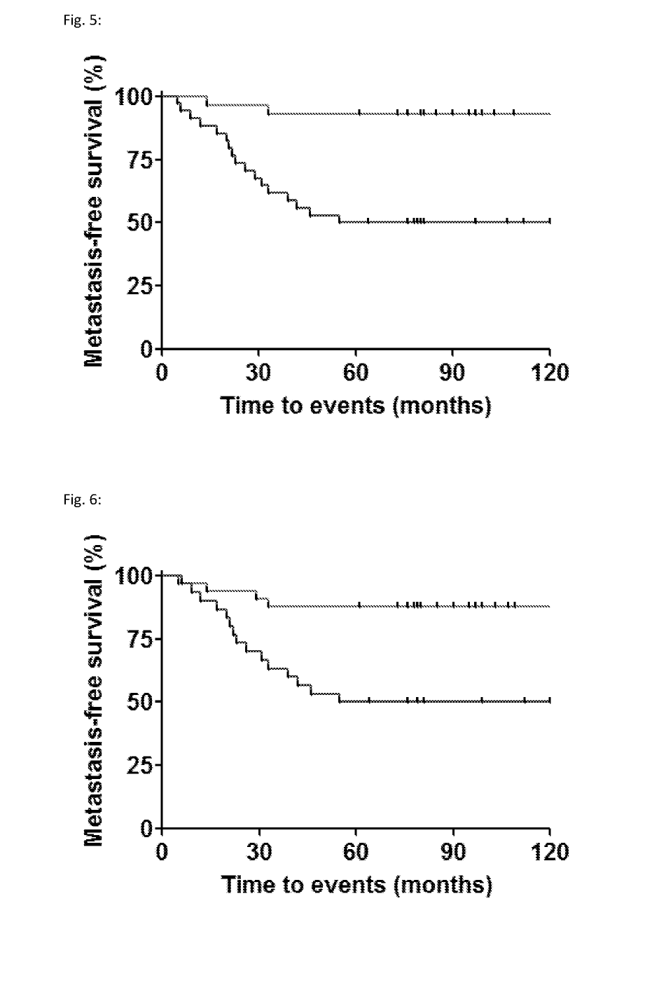

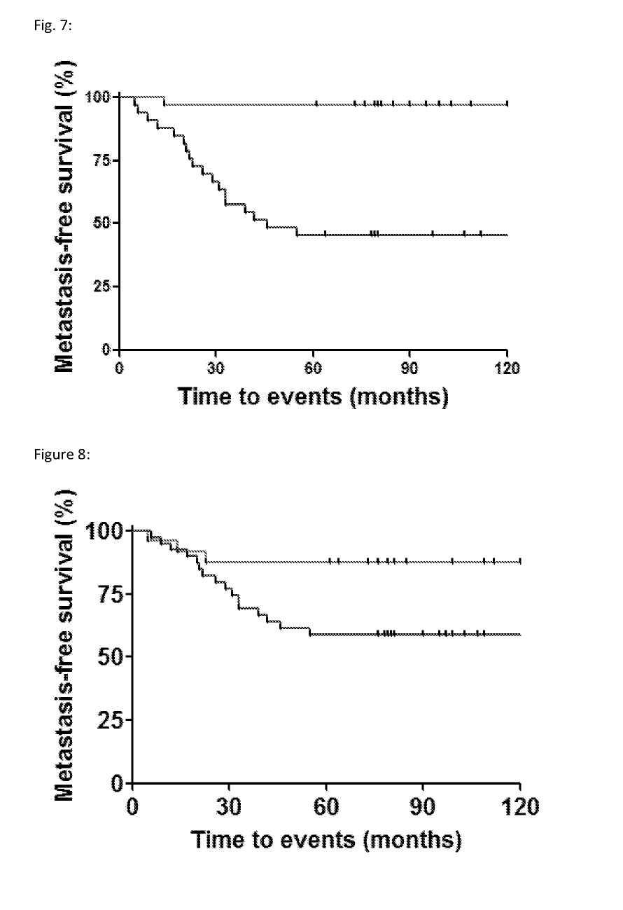

Kaplan Meier curves for survival of different sets of proteins are shown in FIG. 1-X. The set with CMPK1, AIFM1, FTH1, EML4, GANAG, AP1G1, and CAPZB has a sensitivity of more than 90%, see FIG. 1. The model with the highest Youden's index is the set markers with EML4, AP1G1, STX12, and CAPZB, see FIG. 2. The set with EML4, AP1G1, and CAPZB still gives a good prognosis, see FIG. 3. The set with CMPK1, AIFM1, FTH1, AP1G1, AP1M1, CAPZB is shown in FIG. 4. The set with CMPK1, AIFM1, FTH1, AP1G1, CAPZB is shown in FIG. 5. Even the set with only two markers AP1G1 and CAPZB gives a good prognosis, see FIG. 6. Comparison of the set of FIG. 1 without AP1G1 and CAPZB reduces the prognosis results significantly, see FIG. 7. The set with EML4 and STX12 shown in FIG. 8, again showing that a set without AP1G1 and/or CAPZB perform worse.

TABLE 3

| protID | name | cox | p | 95% CI lov | 95% CI hig |
|---|---|---|---|---|---|
| P02794 | FTH1 | −0.44669 | 0 | −0.69533 | −0.19805 |
| P30085 | CMPK1 | −0.60931 | 0 | −0.92391 | −0.29471 |
| O95831 | AIFM1 | −0.91324 | 0.001 | −1.4417 | −0.38477 |
| P11586 | MTHFD1 | 1.259299 | 0.001 | 0.54128 | 1.977318 |
| Q9HC35 | EML4 | −0.56116 | 0.001 | −0.8991 | −0.22321 |
| Q14697 | GANAB | −1.14397 | 0.002 | −1.86169 | −0.42625 |
| O43747 | AP1G1 | −1.02103 | 0.003 | −1.69032 | −0.35174 |
| P35221 | CTNNA1 | −1.11995 | 0.003 | −1.85706 | −0.38284 |
| Q86Y82 | STX12 | −0.7103 | 0.003 | −1.17133 | −0.24926 |
| P47756 | CAPZB | −0.96788 | 0.004 | −1.63067 | −0.30509 |
| Q9BXS5 | AP1M1 | −0.94249 | 0.004 | −1.57516 | −0.30981 |

TABLE 1
Significant expression (66 significant)

| Protein identification | | | | ME-AVONA + T-test | | | | Fisher's exact test + T-test | | | |
|---|---|---|---|---|---|---|---|---|---|---|---|
| Protein | | Gene | | t- | t-test | t-test | | | t-test p | t-test | |
| IDs | UniProt Entry | Name | Protein Name | test | p value | Difference | O | t-test | value | Difference | [symbol] |
| P17612; | KAPCA_HUMAN; | PRKACA; | cAMP-dependent | + | 0.000133704 | 0.593922 | Down | − | — | — | |
| P22694; | KAPCB_HUMAN | PRKACB | protein kinase | | | | | | | | |
| P22612 | | | catalytic | | | | | | | | |
| | | | subunit alpha; | | | | | | | | |
| | | | cAMP-dependent | | | | | | | | |
| | | | protein kinase | | | | | | | | |
| | | | catalytic | | | | | | | | |
| | | | subunit beta | | | | | | | | |
| Q9HC35 | EMAL4_HUMAN | EML4 | Echinoderm | + | 0.000331506 | 0.703975 | Down | − | — | — | |
| | | | microtubule- | | | | | | | | |
| | | | associated | | | | | | | | |
| | | | protein-like 4 | | | | | | | | |
| P30085 | KCY_HUMAN | CMPK1 | UMP-CMP kinase | + | 0.000346956 | 1.03297 | Down | − | — | — | |
| Q14697 | GANAB_HUMAN | GANAB | Neutral alpha- | + | 0.000805477 | 0.625611 | Down | − | — | — | |
| | | | glucosidase AB | | | | | | | | |
| Q9UL46 | PSME2_HUMAN | PSME2 | Proteasome | + | 0.00145094 | 1.13808 | Down | + | 0.00638044 | 1.20117 | [symbol] |
| | | | activator | | | | | | | | |
| | | | complex | | | | | | | | |
| | | | subunit 2 | | | | | | | | |
| P10644; | KAP0_HUMAN | PRKAR1A | cAMP-dependent | + | 0.00162924 | 0.666363 | Down | − | — | — | |
| P31321 | | | protein kinase | | | | | | | | |
| | | | type I-alpha | | | | | | | | |
| | | | regulatory | | | | | | | | |
| | | | subunit | | | | | | | | |
| P02794 | FRIH_HUMAN | FTH1 | Ferritin heavy | + | 0.00202208 | 1.17243 | Down | + | 0.00202208 | 1.17243 | [symbol] |
| | | | chain | | | | | | | | |
| P40925 | MDHC_HUMAN | MDH1 | Malate | + | 0.00207942 | 0.65892 | Down | − | — | — | |
| | | | dehydrogenase, | | | | | | | | |
| | | | cytoplasmic | | | | | | | | |
| Q96FW1 | OTUB1_HUMAN | OTUB1 | Ubiquitin | + | 0.00237328 | 0.533571 | Down | − | — | — | |
| | | | thioesterase | | | | | | | | |
| | | | OTUB1 | | | | | | | | |
| P02787 | TRFE_HUMAN | TF | Serotransferrin | + | 0.0036795 | 0.867287 | Down | − | — | — | |
| Q16555 | DPYL2_HUMAN | DPYSL2 | Dihydro- | + | 0.00401873 | 1.31896 | Down | − | — | — | |
| | | | pyrimidinase- | | | | | | | | |
| | | | related | | | | | | | | |
| | | | protein 2 | | | | | | | | |
| P08493 | MGP_HUMAN | MGP | Matrix Gla | + | 0.00421614 | 1.81668 | Down | − | — | — | |
| | | | protein | | | | | | | | |
| P47756 | CAPZB_HUMAN | CAPZB | F-actin-capping | + | 0.00429814 | 0.360099 | Down | − | — | — | |
| | | | protein | | | | | | | | |
| | | | subunit beta | | | | | | | | |
| P30049 | ATPD_HUMAN | ATP5D | ATP synthase | + | 0.00457028 | 0.486214 | Down | − | — | — | |
| | | | subunit delta, | | | | | | | | |
| | | | mitochondrial | | | | | | | | |
| P23497; | SP100_HUMAN | SP100 | Nuclear | + | 0.00488752 | 0.872863 | Down | − | — | — | |
| Q9H930 | | | autoantigen | | | | | | | | |
| | | | Sp-100 | | | | | | | | |
| Q9UN36 | NDRG2_HUMAN | NDRG2 | Protein NDRG2 | + | 0.00516159 | 1.52476 | Down | + | 0.0117276 | 1.63194 | [symbol] |
| O43169 | CYB5B_HUMAN | CYB5B | Cytochrome b5 | + | 0.00550756 | 0.559582 | Down | − | — | — | |
| | | | type B | | | | | | | | |
| P31948 | STIP1_HUMAN | STIP1 | Stress-induced- | + | 0.0057999 | 0.360835 | Down | − | — | — | |
| | | | phospho- | | | | | | | | |
| | | | protein 1 | | | | | | | | |
| Q9C0C2 | TB182_HUMAN | TNKS1BP1 | 182 kDa | + | 0.00674644 | 0.661646 | Down | − | — | — | |
| | | | tankyrase-1- | | | | | | | | |
| | | | binding protein | | | | | | | | |
| Q9NUQ6 | SPS2L_HUMAN | SPATS2L | SPATS2-like | + | 0.0075694 | 1.3607 | Down | − | — | — | |
| | | | protein | | | | | | | | |
| P14314 | GLU2B_HUMAN | PRKCSH | Glucosidase 2 | + | 0.00854946 | 0.548314 | Down | − | — | — | |
| | | | subunit beta | | | | | | | | |
| P27348 | 1433T_HUMAN | YWHAQ | 14-3-3 protein | + | 0.00881896 | 0.516822 | Down | − | — | — | |
| | | | theta | | | | | | | | |
| Q92896 | GSLG1_HUMAN | GLG1 | Golgi apparatus | + | 0.00963027 | 0.74178 | Down | − | — | — | |
| | | | protein 1 | | | | | | | | |
| P52907 | CAZA1_HUMAN | CAPZA1 | F-actin-capping | + | 0.0104014 | 0.308104 | Down | − | — | — | |
| | | | protein subunit | | | | | | | | |
| | | | alpha-1 | | | | | | | | |
| P15374 | UCHL3_HUMAN | UCHL3 | Ubiquitin | + | 0.011307 | 0.669743 | Down | − | — | — | |
| | | | carboxyl- | | | | | | | | |
| | | | terminal | | | | | | | | |
| | | | hydrolase | | | | | | | | |
| | | | isozyme L3 | | | | | | | | |
| P27797 | CALR_HUMAN | CALR | Calreticulin | + | 0.012084 | 0.523531 | Down | − | — | — | |
| O95747 | OXSR1_HUMAN | OXSR1 | Serine/ | + | 0.0147771 | 0.344608 | Down | − | — | — | |
| | | | threonine- | | | | | | | | |
| | | | protein | | | | | | | | |
| | | | kinase OSR1 | | | | | | | | |
| P38606 | VATA_HUMAN | ATP6V1A | V-type proton | + | 0.0153965 | 0.429488 | Down | − | — | — | |
| | | | ATPase | | | | | | | | |
| | | | catalytic | | | | | | | | |
| | | | subunit A | | | | | | | | |
| P50336 | PPOX_HUMAN | PPOX | Proto- | + | 0.00122662 | −1.24942 | Up | − | — | — | |
| | | | porphyrinogen | | | | | | | | |
| | | | oxidase | | | | | | | | |
| Q8NFF5 | FAD1_HUMAN | FLAD1 | FAD synthase | + | 0.00356988 | −1.03716 | Up | + | 0.0119844 | −0.996079 | |
| P14174 | MIF_HUMAN | MIF | Macrophage | + | 0.00449466 | −0.589365 | Up | − | — | — | |
| | | | migration | | | | | | | | |
| | | | inhibitory | | | | | | | | |
| | | | factor | | | | | | | | |
| P14324 | FPPS_HUMAN | FDPS | Farnesyl | + | 0.00502309 | −0.692774 | Up | + | 0.00502309 | −0.692774 | |
| | | | pyrophosphate | | | | | | | | |
| | | | synthase | | | | | | | | |
| Q8WUY1 | CH055_HUMAN | C8orf55 | UPF0670 protein | + | 0.00735322 | −0.969292 | Up | + | 0.00735322 | −0.969292 | |
| | | | C8orf55 | | | | | | | | |
| Q86UP2 | KTN1_HUMAN | KTN1 | Kinectin | + | 0.00748744 | −0.65075 | Up | − | — | — | |
| Q9BZE4 | NOG1_HUMAN | GTPBP4 | Nucleolar | + | 0.00862551 | −0.58236 | Up | + | 0.00862551 | −0.58236 | |
| | | | GTP-binding | | | | | | | | |
| | | | protein 1 | | | | | | | | |
| Q9H568 | ACTL8_HUMAN | ACTL8 | Actin-like | + | 0.0117905 | −1.57924 | Up | − | — | — | |
| | | | protein 8 | | | | | | | | |
| Q92542 | NICA_HUMAN | NCSTN | Nicastrin | + | 0.0133096 | −0.440308 | Up | − | — | — | — |
| Q9UJZ1 | STML2_HUMAN | STOML2 | Stomatin-like | + | 0.0135585 | −0.496665 | Up | − | — | — | — |
| | | | protein 2 | | | | | | | | |
| Q8NI27 | THOC2_HUMAN | THOC2 | THO complex | + | 0.0136246 | −0.346452 | Up | − | — | — | — |
| | | | subunit 2 | | | | | | | | |
| O60826 | CCD22_HUMAN | CCDC22 | Coiled-coil | + | 0.0150165 | −0.678664 | Up | − | — | — | — |
| | | | domain- | | | | | | | | |
| | | | containing | | | | | | | | |
| | | | protein 22 | | | | | | | | |
| Q562R1 | ACTBL_HUMAN | ACTBL2 | Beta-actin-like | − | — | — | — | + | 0.00264661 | −2.4707 | U [symbol] |
| | | | protein 2 | | | | | | | | |
| P50416 | CPT1A_HUMAN | CPT1A | Carnitine O- | − | — | — | — | + | 0.00447661 | −1.70857 | U [symbol] |
| | | | palmitoyl- | | | | | | | | |
| | | | transferase 1, | | | | | | | | |
| | | | liver isoform | | | | | | | | |
| Q8NFJ5 | RAI3_HUMAN | GPRC5A | Retinoic | − | — | — | — | + | 0.000687272 | −1.42131 | U [symbol] |
| | | | acid-induced | | | | | | | | |
| | | | protein 3 | | | | | | | | |
| Q8NF37 | PCAT1_HUMAN | LPCAT1 | Lyso- | − | — | — | — | + | 0.00194489 | −1.26401 | U [symbol] |
| | | | phosphatidyl- | | | | | | | | |
| | | | choline acyl- | | | | | | | | |
| | | | transferase 1 | | | | | | | | |
| Q9UIJ7 | KAD3_HUMAN | AK3 | GTP: AMP | − | — | — | — | + | 0.0124938 | −1.25993 | U [symbol] |
| | | | phospho- | | | | | | | | |
| | | | transferase, | | | | | | | | |
| | | | mitochondrial | | | | | | | | |
| Q02338 | BDH_HUMAN | BDH1 | D-beta-hydroxy- | − | −−— | — | — | + | 0.0124787 | −1.18236 | U [symbol] |
| | | | butyrate | | | | | | | | |
| | | | dehydrogenase, | | | | | | | | |
| | | | mitochondrial | | | | | | | | |
| Q9UIG0 | BAZ1B_HUMAN | BAZ1B | Tyrosine- | − | — | — | — | + | 0.00967273 | −0.883431 | U [symbol] |
| | | | protein | | | | | | | | |
| | | | kinase BAZ1B | | | | | | | | |
| Q96NB2 | SFXN2_HUMAN | SFXN2 | Sideroflexin-2 | − | — | — | — | + | 0.00710025 | −0.87538 | U [symbol] |
| Q9Y5L0 | TNPO3_HUMAN | TNPO3 | Transportin-3 | − | — | — | — | + | 0.0327844 | −0.796481 | U [symbol] |
| Q16576 | RBBP7_HUMAN | RBBP7 | Histone-binding | − | — | — | — | + | 0.00772753 | −0.668333 | U [symbol] |
| | | | protein RBBP7 | | | | | | | | |
| Q99720 | SGMR1_HUMAN | SIGMAR1 | Sigma | − | — | — | — | + | 0.00435357 | −0.615797 | U [symbol] |
| | | | non-opioid | | | | | | | | |
| | | | intracellular | | | | | | | | |
| | | | receptor 1 | | | | | | | | |
| Q13232 | NDK3_HUMAN | NME3 | Nucleoside | − | — | — | — | + | 0.0143031 | −0.551621 | U [symbol] |
| | | | diphosphate | | | | | | | | |
| | | | kinase 3 | | | | | | | | |
| Q9HB71 | CYBP_HUMAN | CACYBP | Calcyclin- | − | — | — | — | + | 0.0260907 | −0.473641 | U [symbol] |
| | | | binding | | | | | | | | |
| | | | protein | | | | | | | | |
| O75794 | CD123_HUMAN | CDC123 | Cell division | − | — | — | — | + | 0.0263323 | −0.441662 | U [symbol] |
| | | | cycle protein | | | | | | | | |
| | | | 123 homolog | | | | | | | | |
| Q9Y266 | NUDC_HUMAN | NUDC | Nuclear | − | — | — | — | + | 0.0153864 | 0.462346 | Do [symbol] |
| | | | migration | | | | | | | | |
| | | | protein nudC | | | | | | | | |
| P46976 | GLYG_HUMAN | GYG1 | Glycogenin-1 | − | — | — | — | + | 0.0122153 | 0.559155 | [symbol] |
| P52209 | 6PGD_HUMAN | PGD | 6-phospho- | − | — | — | — | + | 0.0266364 | 0.791167 | [symbol] |
| | | | gluconate | | | | | | | | |
| | | | dehydrogenase, | | | | | | | | |
| | | | decarboxylating | | | | | | | | |
| Q9NRN7 | ADPPT_HUMAN | AASDHPPT | L-aminoadipate- | − | — | — | — | + | 0.00346376 | 0.894205 | [symbol] |
| | | | semialdehyde | | | | | | | | |
| | | | dehydrogenase- | | | | | | | | |
| | | | phospho- | | | | | | | | |
| | | | pantetheinyl | | | | | | | | |
| | | | transferase | | | | | | | | |
| Q13190 | STX5_HUMAN | STX5 | Syntaxin-5 | − | — | — | — | + | 0.00307162 | 0.925093 | [symbol] |
| P04080 | CYTB_HUMAN | CSTB | Cystatin-B | − | — | — | — | + | 0.0314616 | 0.925983 | [symbol] |
| P49006 | MRP_HUMAN | MARCKSL1 | MARCKS-related | − | — | — | — | + | 0.00843881 | 1.02378 | [symbol] |
| | | | protein | | | | | | | | |
| Q07954 | LRP1_HUMAN | LRP1 | Prolow-density | − | — | — | — | + | 0.0120908 | 1.11241 | [symbol] |
| | | | lipoprotein | | | | | | | | |
| | | | receptor-related | | | | | | | | |
| | | | protein 1 | | | | | | | | |
| Q06323 | PSME1_HUMAN | PSME1 | Proteasome | − | — | — | — | + | 0.0162815 | 1.16244 | [symbol] |
| | | | activator | | | | | | | | |
| | | | complex | | | | | | | | |
| | | | subunit 1 | | | | | | | | |
| Q96GX9 | MTNB_HUMAN | APIP | Probable | − | — | — | — | + | 0.000607817 | 1.25179 | [symbol] |
| | | | methyl- | | | | | | | | |
| | | | thioribulose-1- | | | | | | | | |
| | | | phosphate | | | | | | | | |
| | | | dehydratase | | | | | | | | |
| P32455 | GBP1_HUMAN | GBP1 | Interferon- | − | — | — | — | + | 0.0129219 | 1.56076 | [symbol] |
| | | | induced | | | | | | | | |
| | | | guanylate- | | | | | | | | |
| | | | binding | | | | | | | | |
| | | | protein 1 | | | | | | | | |
| P54132 | BLM_HUMAN | BLM | Bloom syndrome | − | — | — | — | + | 0.0120878 | 2.0296 | [symbol] |
| | | | protein | | | | | | | | |

[symbol]

indicates data missing or illegible when filed

TABLE 2
protein predictors

| UniProt | | Gene | | | Orientation | Observation |
|---|---|---|---|---|---|---|
| Accession | UniProt Entry | Name | Protein Name | CCV | in Poor | Counts |
| P17612 | KAPCA_HUMAN | PRKACA | cAMP-dependent protein | 4.0781 | down | 63 |
| | | | kinase catalytic subunit | | | |
| | | | alpha | | | |
| P10644 | KAP0_HUMAN | PRKAR1A | cAMP-dependent protein | 3.2977 | down | 63 |
| | | | kinase type I-alpha | | | |
| | | | regulatory subunit | | | |
| O43169 | CYB5B_HUMAN | CYB5B | Cytochrome b5 type B | 2.8781 | down | 63 |
| O43747 | AP1G1_HUMAN | AP1G1 | AP-1 complex subunit | 3.0865 | down | 63 |
| | | | gamma-1 | | | |
| O95831 | AIFM1_HUMAN | AIFM1 | Apoptosis-inducing | 3.2574 | down | 63 |
| | | | factor 1, mitochondrial | | | |
| P02787 | TRFE_HUMAN | TF | Serotransferrin | 3.0208 | down | 63 |
| P02794 | FRIH_HUMAN | FTH1 | Ferritin heavy chain | 3.2256 | down | 63 |
| P14174 | MIF_HUMAN | MIF | Macrophage migration | −2.9506 | up | 63 |
| | | | inhibitory factor | | | |
| P14314 | GLU2B_HUMAN | PRKCSH | Glucosidase 2 subunit | 2.7175 | down | 63 |
| | | | beta | | | |
| P14324 | FPPS_HUMAN | FDPS | Farnesyl pyrophosphate | −2.9111 | up | 63 |
| | | | synthase | | | |
| P23528 | COF1_HUMAN | CFL1 | Cofilin-1 | 2.87 | down | 63 |
| P25786 | PSA1_HUMAN | PSMA1 | Proteasome subunit | 2.7412 | down | 63 |
| | | | alpha type-1 | | | |
| P27348 | 1433T_HUMAN | YWHAQ | 14-3-3 protein theta | 2.706 | down | 63 |
| P30085 | KCY_HUMAN | CMPK1 | UMP-CMP kinase | 3.7904 | down | 63 |
| P31948 | STIP1_HUMAN | STIP1 | Stress-induced- | 2.8595 | down | 63 |
| | | | phosphoprotein 1 | | | |
| P35998 | PRS7_HUMAN | PSMC2 | 26S protease regulatory | 3.0023 | down | 63 |
| | | | subunit 7 | | | |
| P40925 | MDHC_HUMAN | MDH1 | Malate dehydrogenase, | 3.2162 | down | 63 |
| | | | cytoplasmic | | | |
| P47756 | CAPZB_HUMAN | CAPZB | F-actin-capping protein | 2.9664 | down | 63 |
| | | | subunit beta | | | |
| P62820 | RAB1A_HUMAN | RAB1A | Ras-related protein Rab- | 3.7852 | down | 63 |
| | | | 1A | | | |
| Q14697 | GANAB_HUMAN | GANAB | Neutral alpha- | 3.5267 | down | 63 |
| | | | glucosidase AB | | | |
| Q16555 | DPYL2_HUMAN | DPYSL2 | Dihydropyrimidinase- | 2.99 | down | 63 |
| | | | related protein 2 | | | |
| Q562R1 | ACTBL_HUMAN | ACTBL2 | Beta-actin-like protein 2 | −3.1345 | up | 63 |
| Q86UP2 | KTN1_HUMAN | KTN1 | Kinectin | −2.7666 | up | 63 |
| Q8WUY1 | CH055_HUMAN | C8orf55 | UPF0670 protein | −2.7732 | up | 63 |
| | | | C8orf55 | | | |
| Q96FW1 | OTUB1_HUMAN | OTUB1 | Ubiquitin thioesterase | 3.1716 | down | 63 |
| | | | OTUB1 | | | |
| Q9BQE3 | TBA1C_HUMAN | TUBA1C | Tubulin alpha-1C chain | 3.0305 | down | 63 |
| Q9BUJ2 | HNRL1_HUMAN | HNRNPUL1 | Heterogeneous nuclear | 3.0048 | down | 63 |
| | | | ribonucleoprotein U-like | | | |
| | | | protein 1 | | | |
| Q9BZE4 | NOG1_HUMAN | GTPBP4 | Nucleolar GTP-binding | −2.7142 | up | 63 |
| | | | protein 1 | | | |
| Q9COC2 | TB182_HUMAN | TNKS1BP1 | 182 kDa tankyrase-1- | 2.8047 | down | 63 |
| | | | binding protein | | | |
| Q9HC35 | EMAL4_HUMAN | EML4 | Echinoderm | 3.8044 | down | 63 |
| | | | microtubule-associated | | | |
| | | | protein-like 4 | | | |
| P30049 | ATPD_HUMAN | ATP5D | ATP synthase subunit | 2.9465 | down | 62 |
| | | | delta, mitochondrial | | | |
| Q16576 | RBBP7_HUMAN | RBBP7 | Histone-binding protein | −2.721 | up | 62 |
| | | | RBBP7 | | | |
| Q92896 | GSLG1_HUMAN | GLG1 | Golgi apparatus protein 1 | 2.6745 | down | 62 |
| O43865 | SAHH2_HUMAN | AHCYL1 | Putative | 3.9826 | down | 61 |
| | | | adenosylhomocysteinase 2 | | | |
| P68400 | CSK21_HUMAN | CSNK2A1 | Casein kinase II subunit | 2.8616 | down | 61 |
| | | | alpha | | | |
| Q01844 | EWS_HUMAN | EWSR1 | RNA-binding protein | 2.6944 | down | 61 |
| | | | EWS | | | |
| Q9UL46 | PSME2_HUMAN | PSME2 | Proteasome activator | 3.3413 | down | 61 |
| | | | complex subunit 2 | | | |
| P49006 | MRP_HUMAN | MARCKSL1 | MARCKS-related | 2.7601 | down | 60 |
| | | | protein | | | |
| P53990 | IST1_HUMAN | KIAA0174 | IST1 homolog | 2.8383 | down | 60 |
| Q8NFF5 | FAD1_HUMAN | FLAD1 | FAD synthase | −3.0377 | up | 60 |
| Q07000 | 1C15_HUMAN | HLA-C | HLA class I | 3.3356 | down | 59 |
| | | | histocompatibility | | | |
| | | | antigen, Cw-15 alpha | | | |
| | | | chain | | | |
| Q7Z7E8 | UB2Q1_HUMAN | UBE2Q1 | Ubiquitin-conjugating | −2.683 | up | 57 |
| | | | enzyme E2 Q1 | | | |
| P28065 | PSB9_HUMAN | PSMB9 | Proteasome subunit beta | 2.77 | down | 57 |
| | | | type-9 | | | |
| P23497 | SP100_HUMAN | SP100 | Nuclear autoantigen | 2.9352 | down | 56 |
| | | | Sp-100 | | | |
| Q9NUQ6 | SPS2L_HUMAN | SPATS2L | SPATS2-like protein | 2.7749 | down | 56 |
| P35573 | GDE_HUMAN | AGL | Glycogen debranching | 3.0148 | down | 54 |
| | | | enzyme | | | |
| O95249 | GOSR1_HUMAN | GOSR1 | Golgi SNAP receptor | 2.842 | down | 49 |
| | | | complex member 1 | | | |
| Q9UN36 | NDRG2_HUMAN | NDRG2 | Protein NDRG2 | 2.9339 | down | 49 |
| Q05397 | FAK1_HUMAN | PTK2 | Focal adhesion kinase 1 | 3.0059 | down | 46 |
| P08493 | MGP_HUMAN | MGP | Matrix Gla protein | 3.0397 | down | 41 |
| Q9NTJ3 | SMC4_HUMAN | SMC4 | Structural maintenance | −3.1432 | up | 41 |
| | | | of chromosomes protein 4 | | | |
| P50336 | PPOX_HUMAN | PPOX | Protoporphyrinogen | −3.5269 | up | 36 |
| | | | oxidase | | | |
| P10915 | HPLN1_HUMAN | HAPLN1 | Hyaluronan and | −2.8053 | up | 34 |
| | | | proteoglycan link | | | |
| | | | protein 1 | | | |
| Q13190 | STX5_HUMAN | STX5 | Syntaxin-5 | 3.3381 | down | 34 |
| Q15477 | SKIV2_HUMAN | SKIV2L | Helicase SKI2W | 3.474 | down | 33 |
| P09488 | GSTM1_HUMAN | GSTM1 | Glutathione | 2.9203 | down | 28 |
| | | | S-transferase Mu 1 | | | |

1.-44. (canceled)

45. Method for determining a level of expression of a biomarker selected from the group consisting of MTHFD1, CTNNA1, STX12, AP1M1, AP1G1 and/or CAPZB in a biological sample from a patient with triple negative breast cancer

46. Method for treating a patient with triple negative breast cancer comprising determining a level of expression of a biomarker selected from the group consisting of MTHFD1, CTNNA1, STX12, AP1M1, AP1G1 and/or CAPZB in a biological sample from said patient and treating said patient by administering a medicine for triple negative breast cancer.

47. Method according to claim 45 wherein said biomarker is at least one of AP1G1 and/or CAPZB and wherein a further biomarker is selected from the group consisting of MTHFD1, CTNNA1, STX12, and/or AP1M1.

48. Method according to claim 45 wherein a further biomarker is selected from the group consisting of CMPK1, PRKACA, EML4, GANAB, PSME2, PRKAR1A, FTH1 MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, K1AA0174, UBE2Q1, PSMB9, AGL, GOSR1, PTK2, SMC4, HAPLN1, SKIV2L, and/or GSTM1, in a biological sample from said patient.

49. Method according to claim 45 further comprising establishing whether the expression of said biomarker is up-regulated or down-regulated.

50. Method according to claim 45 comparing the level of expression in said sample to a reference level of said biomarker.

51. Method according to claim 45 wherein the level of expression of at least one biomarker selected from the group consisting of MTHFD1, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, UBE2Q1, SMC4, and/or HAPLN1, is up-regulated and/or the level expression of at least one biomarker selected from the group consisting of CTTNA1, STX12, AP1M1, CMPK1, PRKACA; PRKACB, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM is down-regulated in said sample correlates with poor prognosis for said patient.

52. Method according to claim 45 wherein the level expression of at least one biomarker selected from the group consisting of MTHFD1, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, UBE2Q1, SMC4, and/or HAPLN1 is down-regulated and/or the level expression of at least one biomarker selected from the group consisting of CTTNA1, STX12, AP1M1, CMPK1, PRKACA, PRKACB, EML4, GANAB, PSME2, PRKAR1A. FTH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM is up-regulated in said sample correlates with good prognosis for said patient.

53. Method according to claim 45 wherein the effectiveness of treatment for a patient with triple negative breast cancer is determined comprising
determining at a first time point the level of expression at least one biomarker selected from the group comprising CTTNA1, STX12, AP1M1, AIFM1, CMPK1, PRKACA, EML4, GANAB, PSME2, PRKAR1A, FTH1, MDH1, OTUB1, TF, DPYSL2, MOP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1 in a biological sample from said patient
determining at a second time point the level of expression at least one biomarker selected from the group comprising CTTNA1, STX12, AP1M1, AIFM1, CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS. C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1 in a biological sample from said patient.

54. Method according to claim 11 wherein the biomarker at the first and second time point are the same biomarker, and determining the difference in expression level between the first an second time point.

55. Method according to claim 53 wherein the second time point is after treatment is given.

56. Method according to claim 53 wherein no or a small difference in the level of expression of at least one biomarker between the first and second time point is indicative of the effectiveness of the treatment given being low.

57. Method according to claim 53 wherein a difference in the level of expression of at least one biomarker between the first and second time point is indicative of the effectiveness of the treatment given, wherein the level of expression of at least one biomarker selected from the group consisting of MTHFD1, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, UBE2Q1, SMC4, and/or HAPLN1 is higher at the second time point than at the first time point and/or the level expression of at least one biomarker selected from the group consisting of CTTNA1, STX12, AP1M1, CMPK1, PRKACA; PRKACB, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM is lower at the second time point than at the first time point is indicative the effectiveness of the treatment given being low.

58. Method according to claim 53 wherein a difference in the level of expression of at least one biomarker between the first and second time point is indicative of the effectiveness of the treatment given, wherein the level of expression of biomarkers selected from MTHFD1, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7. SIGMAR1, NME3, CACYBP, CDC123, UBE2Q1. SMC4, and/or HAPLN1 is lower at the second time point than at the first time point and/or the level expression of at least one biomarker selected from the group consisting of CTTNA1, STX12, AP1M1, CMPK1, PRKACA; PRKACB, EML4, GANAB, PSME2, PRKAR1A, FTH1, OTUB1, TF, DPYSL2, MGP, CAPZB, ATP5D, SP100, NDRG2, CYB5B, STIP1, TNKS1BP1, SPATS2L. PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM is higher at the second time point than at the first time point, is indicative the effectiveness of the treatment given being high.

59. Method according to claim 45 determining treatment for a patient with triple negative breast cancer comprising determining a level of expression of at least one biomarker selected from the group comprising CTTNA1, STX12, AP1M1, AIFM1, CMPK1, PRKACA, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55. KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1 in a biological sample from said patient.

60. Method according to claim 59 wherein the level expression of at least one biomarker selected from the group consisting of MTHFD1, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, UBE2Q1, SMC4, and/or HAPLN1 is down-regulated and/or the level expression of at least one biomarker selected from the group consisting of CTTNA1, STX12, AP1M1, CMPK1, PRKACA; PRKACB, EML4, GANAB, PSME2, PRKAR1A, FTH1, MDH1, OTUB1, TF, DPYSL2, MGP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM is up-regulated in said sample.

61. Method according to claim 59 wherein the level of expression of at least one biomarker selected from the group consisting of MTHFD1, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, UBE2Q1, SMC4, and/or HAPLN1 is up-regulated and/or the level expression of at least one biomarker selected from the group consisting of CTTNA1, STX12, AP1M1, CMPK1, PRKACA; PRKACB, EML4, GANAB, PSME2, PRKAR1A, FTH1, MDH1, OTUB1, TF, DPYSL2, MOP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, NUDC, GYG1, PGD, AASDHPPT, STX5, CSTB, MARCKSL1, LRP1, PSME1, APIP, GBP1, and/or BLM is down-regulated in said sample.

62. Method according to claim 59 wherein the treatment is selected from the group consisting of chemotherapy, or radiotherapy.

63. Method to screen for compounds for treatment of triple negative breast cancer using at least one biomarker selected from the group consisting of CTTNA1, STX12, AP1M1, AIFM1, CMPK1, PRKACA, EML4, GANAB, PSME2, PRKAR1A, FTH1, MDH1, OTUB1, IF, DPYSL2, MOP, CAPZB, ATP5D, SP100, NDRG2, CYB5B, STIP1, TNKS1BP1, SPATS2L, PRKCSH, YWHAQ, GLG1, CAPZA1, UCHL3, CALR, OXSR1, ATP6V1A, PPOX, FLAD1, MIF, FDPS, C8orf55, KTN1, GTPBP4, ACTL8, NCSTN, STOML2, THOC2, CCDC22, ACTBL2, CPT1A, GPRC5A, LPCAT1, AK3, BDH1, BAZ1B, SFXN2, TNPO3, RBBP7, SIGMAR1, NME3, CACYBP, CDC123, NUDC, GYG1, PGD, AASDHPPT, STX5, CSTB, MARCKSL1, LRP1, PSME1, APIP, GBP1, BLM, AP1G1, AIFM1, CFL1, PSMA1, PSMC2, RAB1A, TUBA1C, HNRNPUL1, AHCYL1, CSNK2A1, EWSR1, KIAA0174, HLA-C, UBE2Q1, PSMB9, AGL, GOSR1, PTK2, SMC4, HAPLN1, SKIV2L, and/or GSTM1.

64. Method according to claim 63 wherein an assay is used that determines the expression level of the biomarker.
